SEQUENCE LISTING (5 sequences)

SEQ ID NO: 1 - DNA, 2201 bp, synthetic construct
  misc_feature 1..2201: note = Synthetic: Malat1p53
  source 1..2201: mol_type = other DNA
gggaacacaacttttaatcatactccagtcgtttcacaataacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccaatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgaggcgcgccttgcctagaaggccggaaaccaactttattactatattccccacaacccccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagacgctacggacttaaataattgagccttaaagaagaaattctttaagtggatgctctcaaactcagggaaacctaaatctagttatagacaaggcaatcctgagccaagccgaagtagtaattagtaagcattctaatagcagcgggatcagaacagtactgcattta

SEQ ID NO: 2 - DNA, 2276 bp, synthetic construct
  misc_feature 1..2276: note = Synthetic: Serapina 1
  source 1..2276: mol_type = other DNA
gggaccagctcaacccttctttaatgtcatccagggagggaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataaggcgcgccttgcctagaaggccggaaaccaactttattactatattccccacaacccccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagacgctacggacttaaataattgagccttaaagaagaaattctttaagtggatgctctcaaactcagggaaacctaaatctagttatagacaaggcaatcctgagccaagccgaagtagtaattagtaaggccagggatggaggggaggggttgaggagcgagaggcag

SEQ ID NO: 3 - DNA, 2201 bp, synthetic construct
  misc_feature 1..2201: note = Synthetic: PSA P53
  source 1..2201: mol_type = other DNA
tgggacaagggccaggaccatctgcaggatgctgtccccgaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccaatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgaggcgcgccttgcctagaaggccggaaaccaactttattactatattccccacaacccccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagacgctacggacttaaataattgagccttaaagaagaaattctttaagtggatgctctcaaactcagggaaacctaaatctagttatagacaaggcaatcctgagccaagccgaagtagtaattagtaatggctcctcacagaggccaccttccaggactactctctga

SEQ ID NO: 4 - DNA, 1826 bp, synthetic construct
  misc_feature 1..1826: note = Synthetic: PSA RDNAse1
  source 1..1826: mol_type = other DNA
tgggacaagggccaggaccatctgcaggatgctgtccccgaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccaccccccaagaagaagagaaaggtgctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtgaggcgcgccttgcctagaaggccggaaaccaactttattactatattccccacaacccccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagacgctacggacttaaataattgagccttaaagaagaaattctttaagtggatgctctcaaactcagggaaacctaaatctagttatagacaaggcaatcctgagccaagccgaagtagtaattagtaatggctcctcacagaggccaccttccaggactactctctga

SEQ ID NO: 5 - DNA, 1826 bp, synthetic construct
  misc_feature 1..1826: note = Synthetic: Malat1 rDNAse
  source 1..1826: mol_type = other DNA
gggaacacaacttttaatcatactccagtcgtttcacaataacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccaccccccaagaagaagagaaaggtgctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtgaggcgcgccttgcctagaaggccggaaaccaactttattactatattccccacaacccccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagacgctacggacttaaataattgagccttaaagaagaaattctttaagtggatgctctcaaactcagggaaacctaaatctagttatagacaaggcaatcctgagccaagccgaagtagtaattagtaagcattctaatagcagcgggatcagaacagtactgcattta

(19) United States
(12) Patent Application Publication
(10) Pub. No.: US 2026/0043045 A1
(43) Pub. Date: Feb. 12, 2026

(54) TARGETED RNA CIRCULARIZATION

(71) Applicant: ESPEROVAX INC., Plymouth, MI (US)

(72) Inventors: David James O'Hagan, Whitmore Lake, MI (US); David Melville, Plymouth, MI (US)

(21) Appl. No.: 19/100,520

(22) PCT Filed: Aug. 1, 2023
(86) PCT No.: PCT/US23/29252

§ 371 Date: Jan. 31, 2025

Related U.S. Application Data
(60) Provisional application No. 63/394,264, filed on Aug. 1, 2022.

(51) Int. Cl. C12N 15/85; C12N 15/88

(52) U.S. Cl. CPC C12N 15/85; C12N 15/88; C12N 2840/203

(57) ABSTRACT

The disclosure provides nucleic acid constructs for cell- and tissue-specific targeting of therapeutic and diagnostic agents.

### CROSS-REFERENCE TO RELATED APPLICATION

This application claims the benefit of priority to U.S. Provisional Application Ser. No. 63/394,264, filed on Aug. 1, 2022, the disclosure of which is expressly incorporated by reference herein in its entirety.

### BACKGROUND

Myriad attempts have been made over the past several decades to express therapeutic and diagnostic nucleic acid payloads in vivo for a wide variety of purposes including, e.g., enzyme replacement, protein supplementation, protein inhibition, vaccination or other immune effector or inhibitor activity, cellular reprogramming, cellular apoptosis or necrosis, epigenetic modification, DNA transcription or intercalation, and the like. Unfortunately, however, despite much promise and some occasional clinical successes, each of delivery, stability, toxicity, specificity and delivery capacity still represent significant technical challenges.

In particular, conventional gene therapy approaches have typically involved the integration of DNA into the genome of one of more transfected host cells, which may allow for longer-lasting expression of the encoded genetic material. The integration of exogenous DNA directly into a host genome may also produce deleterious effects, however, including the inadvertent disruption of endogenous genes and/or aberrant regulation of gene expression. In contrast to DNA, RNA obviates the need for stable integration and/or permanent promoter alterations, but the existing methodologies for RNA-based expression still suffer from one or more of stability, delivery capacity and specificity, with the latter being of primary concern. The present invention addresses these and other unmet needs.

Circular RNAs have been generated for delivering therapeutic proteins into cells, e.g., as described in U.S. Ser. No. 11/352,640 and US2021/0371494. However, the circularization of these RNAs occurs before the molecules are delivered into cells, and accordingly the circularization/payload expression is not cell type-specific. What is needed, then, are methods for the targeted, stable, and cell- or tissue-specific delivery of nucleic acid payloads. Fortunately, the present invention addresses these and other unmet needs.

### SUMMARY OF INVENTION

In one aspect, the disclosure provides a linear RNA polynucleotide for the targeted expression of a payload sequence, the polynucleotide comprising the following elements operably linked to each other and arranged in the following order: (i) a 5′ homology arm, (ii) a 3′ self-splicing intron fragment containing a 3′ splice site dinucleotide, (iii) optionally, a 5′ spacer sequence, (iv) the payload sequence, (v) optionally, a 3′ spacer sequence, (vi) a 5′ self-splicing intron fragment containing a 5′ splice site dinucleotide, and (vii) a 3′ homology arm, wherein the 5′ homology arm and the 3′ homology are complementary to target sequences in a target RNA and are not complementary to each other; and wherein binding of the homology arms to the target RNA allows splicing/ligation at the 3′ and 5′ splice site dinucleotides and production of a circular RNA in a cell or tissue comprising the target RNA.

In embodiments, the self-splicing intron is selected from a group I self-splicing intron, a group II self-splicing intron, and a hammerhead ribozyme. In embodiments, the linear RNA polynucleotide comprises a 5′ spacer sequence, a 3′ spacer sequence, or a 5′ spacer sequence and a 3′ spacer sequence.

In embodiments, the target RNA is a non-coding RNA. In embodiments, the target RNA is a coding RNA, i.e. an mRNA. In embodiments, a target sequence of the target RNA recognized by one or both of the homology arms is located in the 3′ UTR of a mRNA, or overlaps with the 3′ UTR of a mRNA. In embodiments, a target sequence of the target RNA that is recognized by one or both of the homology arms is located in the 5′ UTR of a mRNA, or overlaps with the 5′ UTR of a mRNA. In embodiments, a target sequence of the target RNA that is recognized by one or both of the homology arms is located in a coding sequence of the target RNA, e.g., a mRNA. In embodiments, a target sequence of the target RNA that is recognized by one or both of the homology arms comprises a translation start site. In exemplary embodiments, the target RNA is selected from the group comprising or consisting of actin, insulin, PSA, SerpinA1, and MALAT1.

In embodiments, the payload sequence encodes one or more proteins of interest, e.g. for therapeutic or diagnostic purposes, and the linear RNA polynucleotide further comprises an IRES downstream of the payload sequence. In embodiments, the payload sequence encodes a plurality of proteins, and optionally further comprises protease cleavage sites between the coding sequences for the plurality of proteins.

In embodiments, the therapeutic protein(s) is useful in the treatment of a disease and/or infection, related to a protein deficiency, or able to elicit an immune response for prevention or treatment of disease and/or infection. In exemplary embodiments, the therapeutic protein is selected from the group comprising or consisting of insulin, adalimumab (HUMIRA®), ghrelin, leptin, an alcohol dehydrogenase or other detoxifying enzyme, a cytokine, an anti-microbial protein, a chemokine, a mitogen, an immunogen, a growth factor, and a differentiation factor.

In embodiments, the linear RNA polynucleotide further comprises a muting sequence that inhibits circularization of the linear RNA polynucleotide in absence of the target RNA. In embodiments, the inhibition of circularization is relieved in the presence of the target RNA.

In embodiments, the linear RNA polynucleotide further comprises a riboregulator, or riboswitch. In embodiments, the riboregulator, or riboswitch, functions to selectively inhibit translation of the payload sequence. In some embodiments where the linear RNA polynucleotide comprises a riboregulator, the riboregulator comprises a toehold riboregulator.

In embodiments, the target sequence of a target RNA is located in the 3′ UTR of actin RNA and the payload sequence encodes the tissue regeneration transcription OSK factors (Oct4, Sox2, and Klf4) In an embodiment, the target sequence is located in the 3′ UTR of Malat1 RNA and the payload sequence encodes a p53 protein.

In embodiments, the linear RNA polynucleotide further comprises a nucleic acid binding peptide ligand sequence; optionally wherein the nucleic acid binding peptide ligand sequence is an MS2 ligand sequence.

In another aspect, the disclosure provides a pharmaceutical composition comprising the linear RNA polynucleotide disclosed herein in a pharmaceutically acceptable vehicle.

In another aspect the disclosure provides a DNA construct for production of a linear RNA polynucleotide comprising, operably-linked in the following order: (i) a transcriptional promoter, optionally wherein the transcriptional promoter is a bacteriophage T7 promoter; (ii) a 5′ homology arm, (iii) a 3′ self-splicing intron fragment containing a 3′ splice site dinucleotide, (iv) optionally, a 5′ spacer sequence, (v) a payload sequence, (vi) optionally, a 3′ spacer sequence, (vii) a 5′ self-splicing intron fragment containing a 5′ splice site dinucleotide, and (viii) a 3′ homology arm, wherein the 5′ homology arm and the 3′ homology are complementary to target sequences in a target RNA and are not complementary to each other; and (ix) a transcriptional terminator; optionally wherein the transcriptional terminator is a bacteriophage T7 terminator. In an embodiment, the self-splicing intron is selected from a group I self-splicing intron and a group II self-splicing intron.

In one aspect, the disclosure provides a method of cell-specific expression of a payload sequence in a target cell or tissue, the method comprising: delivering to the target cell or tissue the linear RNA polynucleotide disclosed herein, or a pharmaceutical composition comprising the linear RNA polynucleotide disclosed herein, wherein the target cell or tissue comprises a target RNA.

As demonstrated herein for the first time, hybridization of the homology arms of the linear RNA polynucleotide to target sequences in the target RNA draws the 3′ and 5′ splice site dinucleotides into proximity, thereby facilitating ligation at the splice sites and circularizing the linear RNA polynucleotide, and wherein circularization permits expression of the payload sequence.

In embodiments, the payload sequence encodes one or more proteins of interest, e.g., for therapeutic or diagnostic purposes and the linear RNA polynucleotide further comprises an IRES located downstream of the payload sequence, such that circularization of the linear RNA polynucleotide places/relocates the IRES to a position upstream (5′) of the payload sequence and permits translation of the protein(s) of interest. In embodiments, the payload sequence encodes a plurality of proteins, and optionally further comprises protease cleavage sites between the coding sequences for the plurality of proteins.

In embodiments, the linear RNA polynucleotide is delivered to the cell in vivo or in vitro. In an embodiment, the linear RNA polynucleotide is transfected into the cell by electroporation or lipofection. In an embodiment, the linear RNA polynucleotide is transfected into the cell using a delivery vehicle. In an embodiment, the delivery vehicle is a polymeric carrier; an exosome; a lipid carrier; and/or a lipid nanoparticle. In an embodiment, the linear RNA polynucleotide comprises a nucleic acid binding peptide ligand sequence; optionally wherein the nucleic acid binding peptide ligand sequence is an MS2 ligand sequence, and the delivery vehicle is a yeast cell.

In one aspect, the disclosure provides a method for treating a disease or disorder, the method comprising delivering a payload sequence encoding one or more therapeutic proteins to a target cell or tissue, wherein the payload sequence is present on a linear RNA polynucleotide that comprises at least the following elements, in the following order: (i) a 5′ homology arm, (ii) a 3′ self-splicing intron fragment containing a 3′ splice site dinucleotide, (iii) optionally, a 5′ spacer sequence, (iv) the payload sequence encoding the therapeutic protein(s), (v) optionally, a 3′ spacer sequence, (vi) a 5′ self-splicing intron fragment containing a 5′ splice site dinucleotide, and (vii) a 3′ homology arm, wherein the 5′ homology arm and the 3′ homology are complementary to target sequences in a target RNA and are not complementary to each other; and wherein binding of the homology arms to the target RNA allows splicing/ligation at the 3′ and 5′ splice site dinucleotides and production of a circular RNA in a cell or tissue comprising the target RNA.

In embodiments, hybridization of the homology arms of the linear RNA polynucleotide with the target RNA draws the 3′ and 5′ splice site dinucleotides into proximity, thereby facilitating ligation at the splice sites and circularizing the linear RNA polynucleotide, wherein circularization permits expression of the payload sequence.

In embodiments, the payload sequence encodes one or more therapeutic proteins of interest, and the linear polynucleotide further comprises an IRES downstream (3′) of the payload sequence such that circularization of the linear RNA polynucleotide places/relocates the TRES to a position upstream (5′) of the payload sequence and permits translation of the therapeutic protein. In embodiments, the payload sequence encodes a plurality of proteins, and optionally further comprises protease cleavage sites between the coding sequences for the plurality of proteins.

In embodiments, the linear RNA polynucleotide further comprises a muting sequence that inhibits circularization of the linear RNA polynucleotide in a cell or tissue lacking the target RNA.

In embodiments, the linear RNA polynucleotide may comprise a riboregulator or riboswitch that functions to selectively inhibit or allow translation of the payload sequence.

Other features objects and advantages will be apparent from the disclosure that follows.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates an exemplary DNA construct for producing a linear RNA polynucleotide as disclosed herein.

FIG. 2 illustrates Targeted RNA Circularization using group 1 introns as disclosed herein.

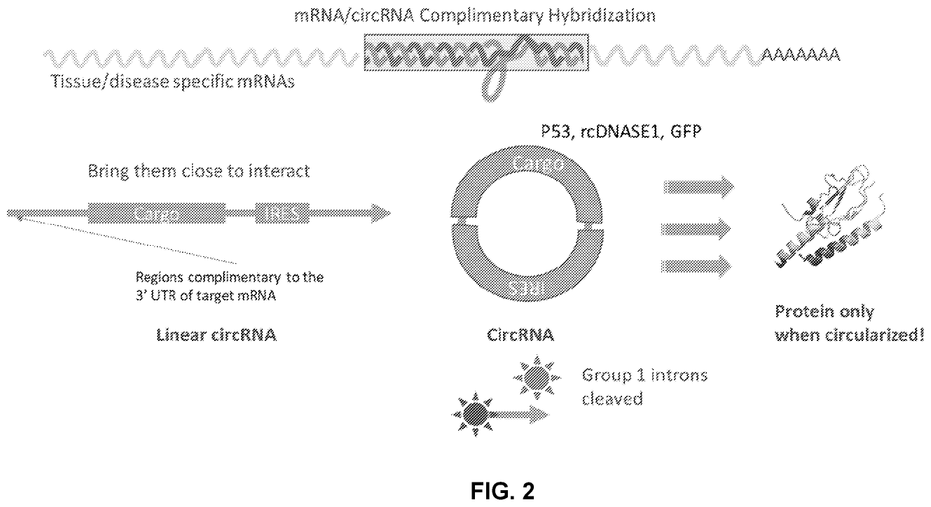

FIG. 3 illustrates regulation of metastasis-associated lung adenocarcinoma transcript 1 (MALAT1).

FIG. 4 illustrates a DNA construct for producing a linear RNA polynucleotide useful for inducing apoptosis of metastatic cancer cells using Malat1 as target RNA, as disclosed in Example 2, herein below.

FIG. 5 illustrates Liver Specific protein replacement using defective Serpina1 as target RNA.

FIG. 6 illustrates Prostate Specific Apoptosis Induction of Cancer cells using PSA as target RNA.

FIG. 7 illustrates Metastatic Enriched Apoptosis Induction of Metastatic Cancer cells using Malat1 as target RNA.

FIG. 8A shows that the linear RNA polynucleotide circularized in the presence of PSA mRNA. FIG. 8B shows that the linear RNA polynucleotide selectively circularized in prostate cancer cells but not breast cancer cells.

### DETAILED DESCRIPTION

Targeted RNA circularization and expression of a payload sequence using linear RNA polynucleotides comprising self-splicing introns are disclosed herein. The linear RNA polynucleotide disclosed herein is useful for cell-specific targeting and delivery of payload sequences encoding therapeutic proteins or peptides. The linear RNA polynucleotide for the targeted expression of a payload sequence comprises at least the following elements operably-linked to each other and arranged in the in the following order: (i) a 5′ homology arm, (ii) a 3′ self-splicing intron fragment containing a 3′ splice site dinucleotide, (iii) optionally, a 5′ spacer sequence, (iv) the payload sequence, (v) optionally, a 3′ spacer sequence, (vi) a 5′ self-splicing intron fragment containing a 5′ splice site dinucleotide, and (vii) a 3′ homology arm, wherein the 5′ homology arm and the 3′ homology arm are complementary to target sequences in a target RNA and are not complementary to each other; and wherein binding of the homology arms to the target RNA allows splicing/ligation at the 3′ and 5′ splice site dinucleotides and production of a circular RNA in a cell or tissue comprising the target RNA.

As demonstrated herein, the cell-specific targeting contemplated by the subject invention is facilitated by “homology arms” located at the 5′ and 3′ ends of the linear RNA polynucleotide. These homology arms are designed to hybridize with target sequences in a tissue-specific or cell-specific RNA, as opposed to hybridizing with each other. In embodiments, the target sequences may be adjacent one another in the target RNA. In embodiments, there may be a gap between target sequences in the target RNA, with each target sequence recognized by a homology arm. In some embodiments, the gap is one nucleotide, or greater than one nucleotide, for example 2, 3, 4, 5, 6, 7, 8, 9, 10, 11, 12, 13, 14, 15, 16, 17, 18, 19, or 20 nucleotides. In some embodiments, the gap between target sequences in a target RNA may be larger, for example when the target RNA exhibits a structure wherein target sequences are positioned proximate one another in space but not necessarily along the length of the target RNA. Hybridization of the homology arms with the tissue or cell specific target RNA brings the self-splicing introns and their dinucleotide splice sites into proximity such that they can splice together at the dinucleotide splice site, thereby circularizing the RNA. Circularization stabilizes the RNA.

In embodiments, the linear RNA polynucleotide comprises a payload sequence encoding one or more proteins of interest, and further comprises an IRES downstream (3′) of the payload sequence. In such embodiments, circularization places the IRES in position upstream (5′) of the payload sequence where it can facilitate translation. Thus, the linear polynucleotides disclosed herein can simultaneously target and concentrate the delivery of one or more therapeutic proteins in cell populations where they are needed, thereby reducing side effects of therapy.

Delivery of the linear RNA polynucleotide to a cell or tissue allows for targeted expression of the payload sequence. Thus, in embodiments, the disclosure provides methods for treating a disease or disorder by tissue- or cell-specific expression of the payload sequence.

In some embodiments, the linear RNA polynucleotides produce one or more therapeutic proteins only in cells naturally equipped to process these proteins, thus reducing off-target effects and optimizing therapeutic benefits; e.g., expressing insulin in pancreatic R cells, the natural site of insulin production, or expressing GLP-1 in intestinal L cells, the natural site of GLP-1 production, thereby harnessing and mimicking the body's natural production. Such approaches can improve safety and efficacy over existing treatments.

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

As used herein, “about” is understood by persons of ordinary skill in the art and may vary to some extent depending upon the context in which it is used. The term “about” indicates and encompasses an indicated value and a range above and below that value. In certain embodiments, the term “about” indicates the designated value±10%, ±5%, or ±1%. In certain embodiments, where indicated, the term “about” indicates the designated value±one standard deviation of that value.

As will be understood by one skilled in the art, for any and all purposes, all ranges disclosed herein also encompass any and all possible subranges and combinations of subranges thereof. Furthermore, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

The term “combinations thereof” includes every possible combination of elements to which the term refers.

The term “subject” or “patient” as used herein, refers to an individual or mammal having a disease or at elevated risk of having a disease (e.g., having or at elevated risk of having diabetes or cancer). The subject may be any mammal, including both a human and other mammals, e.g. an animal such as a rabbit, mouse, rat, or monkey. Human subjects are preferred.

The term “operably-linked” or “operably linked” as used herein, refers to polynucleotide sequence(s) that are functionally connected. Elements of a linear RNA polynucleotide are operably-linked when they are positioned along a linear RNA polynucleotide such that they are able to circularize to provide a circular RNA according to the methods provided herein. For example, a “linear RNA polynucleotide” comprises at least the following elements “operably-linked” in the following order: (i) a 5′ homology arm, (ii) a 3′ group I intron fragment containing a 3′ splice site dinucleotide, (iii) optionally, a 5′ spacer sequence, (iv) a payload sequence, (v) optionally, a 3′ spacer sequence, (vi) a 5′ group I intron fragment containing a 5′ splice site dinucleotide, and (vii) a 3′ homology arm, wherein the 5′ homology arm and the 3′ homology are complementary to target sequences in a target RNA and are not complementary to each other.

The term “homology arm” as used herein, refers to a contiguous sequence at least 5 nucleotides long and no longer than 250 nucleotides that forms or is predicted to form base pairs with at least about 75% (e.g., at least about 80%, at least about 85%, at least about 90%, at least about 95%, about 100%) of a complementary target sequence in a target RNA. Typically, a “homology arm” exhibits less than 50% (e.g., less than 45%, less than 40%, less than 35%, less than 30%, less than 25%) base pairing with unintended sequences in the target RNA and/or with unintended sequences in the linear RNA polynucleotide itself 5′ and 3′ homology arms are not complementary with each other.

The term “target RNA” as used herein, refers to a contiguous nucleic acid sequence at least 1 nucleotide long, which may be coding or non-coding, that base pairs with at least about 75% (e.g., at least about 80%, at least about 85%, at least about 90%, at least about 95%, about 100%) of a “homology arm” of the linear RNA polynucleotide disclosed herein. Target RNA will generally be located in a cell or tissue of interest for therapeutic and/or diagnostic purposes.

The term “complementary” as used herein, has the meaning commonly understood in the art and refers to the ability of a nucleic acid to form hydrogen bond(s) by either traditional Watson-Crick base-pairing or other nontraditional type base-pairing. In reference to the nucleic acid molecules disclosed herein, the binding free energy for a nucleic acid molecule with its complementary sequence is sufficient to allow the relevant function of the nucleic acid to proceed, e.g., tissue and/or cell specific targeting of a linear RNA polynucleotide comprising a payload sequence encoding a therapeutic protein. Determination of binding free energies for nucleic acid molecules is well-known in the art (see, e.g., Turner et al, 1987, CSH Symp. Quant. Biol., 1987, LII, pp 123-133, Frier et al, P.N.A.S., 1986, 83, 9373-9377; Turner at al., J Am. Chem. Soc, 1987, 109, 3783-3785). A percent complementarity indicates the percentage of contiguous residues in a nucleic acid molecule that can form hydrogen bonds (e.g., Watson-Crick base-pairing) with a second nucleic acid sequence (e.g., 5, 6, 7, 8, 9, or 10 nucleotides out of a total of 10 nucleotides, in the first oligonucleotide being base paired to a second nucleic acid sequence having 10 nucleotides represents 50%, 60%, 70%, 80%, 90% and 100% complementarity, respectively). “Perfectly complementary” means that all the contiguous residues of a nucleic acid sequence will hydrogen bond with the same number of contiguous residues in a second nucleic acid sequence.

The term “melting temperature” or “Tm,” as used herein has the meaning commonly known and understood in the art and refers to the temperature at which 50% of the nucleic acid molecules in a solution are single-stranded (SS) while 50% of the molecules are in the double-stranded (DS) form. Typically, melting temperature increase as the guanine-cytosine (GC) content of the nucleic acid duplex increases. “Tm” is readily determined according to methods known in the art, see e.g., Dumousseau, M., et al. MELTING, a flexible platform to predict the melting temperatures of nucleic acids. BMC Bioinformatics 13, 101 (2012). Typically, the Tm of duplexes formed between a homology arm and a target sequence are optimized to promote circularization of the liner RNA polynucleotide in a target cell.

The term “coding sequence” as used herein has the meaning commonly known and understood in the art and refers to a nucleic acid sequence e.g., an RNA sequence, that encodes a polypeptide or protein. Similarly, the term “non-coding sequence” as used herein has the meaning commonly known in the art and refers to a nucleic acid sequence e.g., an RNA sequence, that does not encode a polypeptide or protein.

The term “self-splicing intron” as used herein, refers to introns that form a ribozyme which is able to perform the functions of the spliceosome by RNA alone, without the participation of any proteins. Group I and Group II introns are examples of self-splicing introns, and are well known in the art (see e.g., Clancy, S. (2008) RNA splicing: introns, exons and spliceosome. Nature Education 1(1):31, as well as hammerhead ribozymes, which are small catalytic RNA motifs capable of endonucleolytic (self-) cleavage. Suitable examples of hammerhead ribozymes (HHR) for use as self-splicing introns in the subject invention include those described in Pena M. et al., Circular RNAs with hammerhead ribozymes encoded in eukaryotic genomes: The enemy at home, RNA Biol. 2017; 14(8): 985-991, which is incorporated by reference herein in its entirety.

The term “3′ self-splicing intron or fragment thereof” refers to a sequence with 75% or higher (e.g., at least 80%, at least 85%, at least 90%, at least 95%, 100%) similarity to the 3′-proximal end of a natural group I, group II or HHR intron including the splice site dinucleotide and optionally a stretch of natural exon sequence. The adjacent exon sequence is typically at least 1 nucleotide in length (e.g., at least 5 nucleotides in length, at least 10 nucleotides in length, at least 15 nucleotides in length, at least 20 nucleotides in length, at least 25 nucleotides in length, at least 50 nucleotides in length). In one embodiment, the included adjacent exon sequence is about the length of the natural exon.

The term “5′ self-splicing intron fragment thereof” refers to a sequence with 75% or higher (e.g., at least 80%, at least 85%, at least 90%, at least 95%, 100%) similarity to the 5′-proximal end of a natural group I, group II or HHR intron including the splice site dinucleotide and optionally a stretch of natural exon sequence. The adjacent exon sequence is typically at least 1 nucleotide in length (e.g., at least 5 nucleotides in length, at least 10 nucleotides in length, at least 15 nucleotides in length, at least 20 nucleotides in length, at least 25 nucleotides in length, at least 50 nucleotides in length). In one embodiment, the included adjacent exon sequence is about the length of the natural exon.

As used herein, the term “splice site” refers to a dinucleotide that is partially or fully included in a self-splicing (e.g., group I, group II or HHR) intron and between which a phosphodiester bond is cleaved during RNA circularization. Thus, a “splice site dinucleotide” refers to the dinucleotide or dinucleotides between which cleavage of the phosphodiester bond occurs during a splicing reaction. A “5′ splice site” refers to the natural 5′ dinucleotide of the intron e.g., group I intron, while a “3′ splice site” refers to the natural 3′ dinucleotide of the intron.

As used herein, a “spacer” refers to a region of a contiguous polynucleotide sequence ranging from 1 nucleotide to hundreds or thousands of nucleotides separating two other elements along a polynucleotide sequence. The sequences can be defined or can be random. A spacer is typically non-coding. In some embodiments, spacers include duplex forming regions. Typically, a spacer sequence is used to prevent interference between proximal structures that may alter the folding of structures in the RNA, such as the IRES or self-splicing intron-derived sequences. Spacer sequences are typically at least 7 nucleotides long (and optionally no longer than 100 nucleotides) and located downstream of and adjacent to the 3′ intron fragment and/or upstream of and adjacent to the 5′ intron fragment; and/or contains one or more of the following: a) an unstructured region at least 5 nucleotides long b) a region predicted base pairing at least 5 nucleotides long to a distal (i.e., non-adjacent) sequence, including another spacer, and/or c) a structured region at least 1 nucleotide long limited in scope to the sequence of the spacer.

The term “PIE” or “permutated intron-exon” as used herein refers to a spontaneous group I intron self-splicing system, designated as the permuted intron-exon (PIE) method, which is known in the art (see e.g., Puttaraju & Been, (1992) Nuc. Acids Res. 20:5357-5364). The PIE method comprises inverting the location of the 3′ and 5′ intron on a linear RNA polynucleotide such that the 3′ end of the exon is joined to a splice site at an upstream rather than a downstream position (donor (5′) splice site is 3′ of the acceptor (3′) splice site) thereby permitting formation of a circular RNA. Using Group I self-splicing permuted intron-exon (PIE) sequences in which the order of the splice sites has been reversed, it is possible to catalyze the circularization of a variety of RNA exon sequences in vitro, as well as in vivo in bacteria and in yeast (Puttaraju, M. and Been, (1992) supra, and GenBank X69005). Circular RNAs generated by splicing have been demonstrated with in vitro manipulated Group I and Group II intron sequences.

The term “protein” as used herein refers broadly to a polymer of amino acid residues of any length, including peptides and polypeptides, and encompasses therapeutic proteins that can be used to promote well-being of humans or animals, as well as diagnostic and/or reporter proteins as disclosed herein. A therapeutic protein can be a medicine, an antibody, an antigen, a vaccine, insulin, a nutritional supplement, etc. Therapeutic proteins can also be proteins that are an intermediate in the biosynthetic pathway of a protein that is ultimately used to promote well-being of humans or animals. The terms apply to amino acid polymers in which one or more amino acid residue is an artificial chemical mimetic of a corresponding naturally occurring amino acid, as well as to naturally occurring amino acid polymers and non-naturally occurring amino acid polymers. The term “recombinant protein” refers to a peptide or polypeptide that is produced by recombinant techniques, wherein generally DNA or RNA encoding the expressed protein is inserted into a suitable expression vector that is turn used to transform a host cell to produce the polypeptide. In some exemplary embodiments, DNA or RNA encoding an expressed peptide or polypeptide is inserted into the host chromosome via homologous recombination or other means well known in the art, and is so used to transform a host cell to produce the peptide or polypeptide. Similarly, the terms “recombinant polynucleotide” or “recombinant nucleic acid” or “recombinant DNA” are produced by recombinant techniques that are known to those of skill in the art (see e.g., Sambrook et al., supra, Current Protocols in Molecular Biology, supra).

The term “payload sequence” as used herein, refers broadly to a nucleic acid sequence that encodes an amino acid-based polymer of interest, e.g., for therapeutic or diagnostic purposes, the targeted expression of which is desired and enabled by the subject invention.

The term “host microorganism” as used herein, refers generally to a microscopic organism. Microorganisms can be prokaryotic or eukaryotic. Exemplary prokaryotic microorganisms include e.g. bacteria, archaea, cyanobacteria, etc. A host microorganism can also be eukaryotic, such as a fungus. An exemplary fungus is the yeast, e.g., Saccharomyces cerevisiae. In exemplary embodiments, a “recombinant microorganism” is a microorganism that has been genetically altered and thereby expresses or encompasses a heterologous nucleic acid sequence and/or a heterologous protein. A “host microorganism” or equivalently a “host cell” is a cell used to produce products. As disclosed herein, a “host microorganism” is typically modified to express or overexpress selected genes, or to have attenuated expression of selected genes. Thus, a “host microorganism” or a “host cell” is a “recombinant host” or equivalently a “recombinant host cell.”

“Expression control sequences” or “regulatory sequences” are known in the art and include, for example, promoters, enhancers, polyadenylation signals, transcription terminators, nucleotide sequences that affect RNA stability, internal ribosome entry sites (IRES), and the like, that provide for the expression of the polynucleotide sequence in a host cell. In exemplary embodiments, “expression control sequences” interact specifically with cellular proteins involved in transcription (see e.g., Maniatis et al, Science, 236: 1237-1245 (1987); Goeddel, Gene Expression Technology: Methods in Enzymology, Vol. 185, Academic Press, San Diego, Calif (1990)). In exemplary methods, an expression control sequence is operably linked to a polynucleotide sequence.

The term “regulatable promoter” as used herein refers to a region of DNA that initiates transcription of a particular gene under specific conditions. The term includes inducible promoters and repressible promoters. Examples of inducible promoters include both positive inducible promoters, i.e., inducible promoters that are activated in the presence of the inducer, such as by interaction between the inducer and an activator molecule to enable binding of the combined entity to the inducible promoter to effect transcription of downstream genes controlled by the inducible promoter, and negative inducible promoters, i.e., inducible promoters that are activated in the presence of the inducer, such as by interaction between the inducer and a repressor to block or disable binding of the repressor to the inducible promoter, thereby removing suppression of transcription of downstream genes controlled by the inducible promoter. Examples of repressible promoters include both positive repressible promoters, i.e., promoters that are repressed in the presence of the repressor, such as by interaction between the repressor and an activator molecule to block or disable binding of the activator molecule to the repressible promoter, thereby removing activation of transcription of downstream genes controlled by the repressible promoter, and negative repressible promoters, i.e., promoters that are repressed in the presence of the repressor, such as by interaction between the repressor and a corepressor molecule to enable binding of the combined entity to the repressible promoter to effect transcription of downstream genes controlled by the repressible promoter. The term also includes promoters that can be regulated as both a positive inducible promoter and a negative inducible promoter, and promoters that respond to environmental queues, such as the presence or absence of light, the absence of a particular molecule, and any other promoter that can be specifically regulated by providing or removing a particular molecule or environmental queue.

The term “transcriptional promoter” as used herein, has the meaning commonly known in the art and refers to a region of DNA where transcription of a gene is initiated.

The term “transcriptional terminator” as used herein, has the meaning commonly known in the art and refers to a section of nucleic acid sequence that marks the end of a gene or operon in genomic DNA during transcription. This sequence mediates transcriptional termination by providing signals in the newly synthesized transcript RNA that trigger processes which release the transcript RNA from the transcriptional complex.

The term “nucleic acid binding peptide ligand sequence” as used herein, refers to a nucleic acid sequence capable of binding (or being bound by) nucleic acid binding peptide. In an exemplary embodiment, the nucleic acid binding peptide comprises an MS2 peptide sequence, and the nucleic acid binding peptide ligand sequence comprises an MS2 ligand sequence.

The term “muting sequence” as used herein, refers to a ribonucleic acid (RNA) sequence included as part of a linear RNA polynucleotide as herein disclosed that recognizes and can pair via Watson-Crick base pairing with a sequence that is part of a splicing intron fragment included in the linear RNA polynucleotide. When the muting sequence is paired with its complementary sequence comprising a desired portion of a self-splicing intron fragment, the pairing may prevent splicing/ligation at the 3′ and 5′ splice site dinucleotides thereby preventing the production of a circular RNA. The inhibitory nature of the muting sequence may be relieved by the homology arm(s) of the linear RNA polynucleotide recognizing their respective complementary target sequences in the target RNA, whereby the binding of the homology arm(s) to the target RNA induces a change (e.g., conformational change) that disrupts the binding of the muting sequence to the complementary sequence that is part of a splicing intron fragment. Once the binding of the muting sequence to the complementary sequence has been disrupted, circularization of the RNA may proceed. In embodiments, the muting sequence may have a stronger affinity for its counterpart complementary sequence that is part of the linear RNA polynucleotide in the absence of the target RNA, whereas in the presence of the target RNA the homology arm(s) hybridize with the target RNA which reduces the affinity of the muting sequence for the complementary sequence that is part of the linear RNA polynucleotide.

The term “riboregulator” as used herein, refers to ribonucleic acid (RNA) that responds to a signal nucleic acid molecule by Watson-Crick base pairing. Riboregulators respond to a signal molecule in any number of ways and may function to facilitate and/or block translation of an RNA into a protein, can activate a ribozyme, release a silencing RNA (siRNA), induce conformational change, and/or bind other nucleic acids. Riboregulators can be tailored to respond to complex biological signals, and thus can be configured to respond to tissue and cell specific signals e.g., by sensing and responding to the presence of a disease specific RNA. Riboregulators are known in the art (see e.g., Krishnamurthy et al. ACS Synth Biol. 2015 Dec. 18; 4(12): 1326-1334; U.S. Pat. Nos. 9,550,987; 11,124,846).

Riboregulators contain two canonical domains, a sensor domain and an effector domain. The sensor domain only binds complementary RNA or DNA strands based on base-pairing, and therefore can differentiate and respond to individual genetic sequences and combinations thereof.

The term “riboswitch” as used herein, refers to a regulatory segment of a messenger RNA molecule that binds a small molecule, resulting in a change in production of the proteins encoded by the mRNA. An mRNA that contains a riboswitch is directly involved in regulating its own activity, in response to the concentrations of its effector molecule. Like riboregulators riboswitches contain two canonical domains, a sensor domain and an effector domain. However, a riboswitch sensor domain binds a small molecule instead of complementary RNA or DNA strands.

As used herein, an “internal ribosome entry site” or “IRES” refers to an RNA sequence or structural element ranging in size from 10 nucleotides to 1000 nucleotides or more, capable of initiating translation of a polypeptide in the absence of a typical RNA cap structure. An TRES is typically about 500 nucleotides to about 700 nucleotides in length.

As used herein, the term “translation efficiency” refers to a rate or amount of protein or peptide production from a ribonucleotide transcript. In some embodiments, translation efficiency can be expressed as amount of protein or peptide produced per given amount of transcript that codes for the protein or peptide.

The terms “duplexed,” “double-stranded” or “hybridized” as used herein refer to nucleic acids formed by hybridization of two single strands of nucleic acids containing complementary sequences. In most cases, genomic DNA is double-stranded. Sequences can be fully complementary or partially complementary.

The term “biomarker” as used herein, refers to a characteristic that can be objectively measured and evaluated as an indicator of normal and disease processes or pharmacological responses. A “biomarker” is a parameter that can be used to measure the onset or the progress of disease or the effects of treatment. The parameter can be chemical, physical or biological.

The phrase “lipid nanoparticle” as used herein, refers to a delivery vehicle comprising one or more lipids (e.g., in some embodiments, cationic lipids, non-cationic lipids, and PEG-modified lipids).

The term “disease,” or “disease or disorder” as used herein, refers any condition or disorder that damages or interferes with the normal function of a cell, tissue, or organ. Examples of diseases include neoplasia, pathogen infection of cell, etc.

As used herein, “treating” or “treatment” of any disease or disorder refers, in certain embodiments, to ameliorating a disease or disorder that exists in a subject. “Treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder. For example, in an exemplary embodiment, the phrase “treating cancer” refers to inhibition of cancer cell proliferation, inhibition of cancer spread (metastasis), inhibition of tumor growth, reduction of cancer cell number or tumor growth, decrease in the malignant grade of a cancer (e.g., increased differentiation), or improved cancer-related symptoms. Further, as used herein, “treatment” includes preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer.

As used herein, the term “therapeutically effective amount” or “effective amount” refers to an amount of a substance of a composition comprising a substance e.g., a therapeutic protein, or therapeutic nucleic acid, that when administered to a subject is effective to treat a disease or disorder. For example, in an exemplary embodiment, the phrase “effective amount” is used interchangeably with “therapeutically effective amount” or “therapeutically effective dose” and the like, and means an amount of a therapeutic agent that is effective to prevent or ameliorate a disease or the progression of the disease e.g., cancer, or result in amelioration of symptoms. Effective amounts of the compositions provided herein may vary according to factors such as the disease state, age, sex, weight of the animal, or human.

The term “pharmaceutical composition,” as used herein, refers to a composition that can be administrated to a subject in the context of treatment of a disease or disorder. In some embodiments, a pharmaceutical composition comprises an active ingredient, e.g., therapeutic protein(s) as disclosed herein, and a pharmaceutically acceptable excipient.

Unless defined otherwise, technical and scientific terms used herein have the same meaning as commonly understood by a person of ordinary skill in the art. In particular, this disclosure utilizes routine techniques in the field of recombinant genetics, immunology, and biochemistry. Basic texts disclosing the general terms in molecular biology and genetics include e.g., Lackie, Dictionary of Cell and Molecular Biology, Elsevier (5th ed. 2013). Basic texts disclosing methods in recombinant genetics and molecular biology include e.g., Sambrook et al, Molecular Cloning—A Laboratory Manual, Cold Spring Harbor Press 4th Edition (Cold Spring Harbor, N.Y. 2012) and Current Protocols in Molecular Biology Volumes 1-3, John Wiley & Sons, Inc. (1994-1998) and Supplements 1-115 (1987-2016). Basic texts disclosing the general methods and terms in biochemistry include e.g., Lehninger Principles of Biochemistry sixth edition, David L. Nelson and Michael M. Cox eds. W.H. Freeman (2012). Basic texts disclosing the general methods and terms immunology include Janeway's Immunobiology (Ninth Edition) by Kenneth M. Murphy and Casey Weaver (2017) Garland Science; Fundamental Immunology (Seventh Edition) by William E. Paul (2013) Lippincott, Williams and Wilkins.

The disclosure provides methods and compositions for the targeted expression of a desired payload sequence in vivo and in vitro. As will be clear from the disclosure that follows, several features of the linear RNA polynucleotide facilitate tissue specific targeting and expression.

### I. Linear RNA Polynucleotides

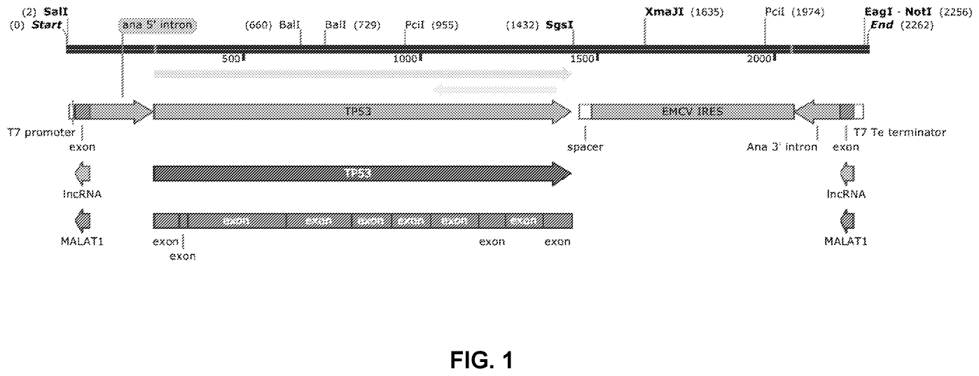

In embodiments, a DNA construct for producing a linear RNA construct comprises at least the following elements arranged in the following sequence (i) a transcriptional promoter; (ii) a 5′ target homology arm; (iii) a 3′ self-splicing group I intron fragment comprising a 3′ splice site dinucleotide; (iv) optionally, a 5′ spacer sequence; (v) a payload sequence; (vi) an internal ribosome entry site (IRES); (vii) optionally, a 3′ spacer sequence; (viii) a 5′ self-splicing group I intron fragment comprising a 5′ splice site dinucleotide; and (ix) a 3′ target homology arm wherein the 5′ homology arm and the 3′ homology are not complementary to each other. An example of the DNA construct is shown in FIG. 1.

In embodiments, the linear RNA polynucleotide produced by transcription of the DNA construct is designed to permit and facilitate expression of the payload sequence only in a target tissue or cell of interest and comprises the following elements arranged in the following sequence (i) a 5′ homology arm, (ii) a 3′ group I intron fragment containing a 3′ splice site dinucleotide, (iii) optionally, a 5′ spacer sequence, (iv) a payload sequence, (v) an IRES; (vi) optionally, a 3′ spacer sequence, (vii) a 5′ group I intron fragment containing a 5′ splice site dinucleotide, and (viii) a 3′ homology arm, wherein the 5′ homology arm and the 3′ homology are complementary to a target RNA and are not complementary to each other.

In embodiments, the linear RNA polynucleotide produced by transcription of the DNA construct comprises elements such as the homology arms, for targeting disease, cell, or tissue specific RNA, and further comprises a payload sequence of interest. In some embodiments, the payload sequence encodes a therapeutic protein. In some embodiments, the linear RNA polynucleotide comprises regulatory elements that specifically regulate expression of the payload sequence. The linear RNA polynucleotide may also comprise elements that facilitate circularization of the linear polynucleotide in a desired cell targeted by the homology arms. Circularization of the linear RNA polynucleotide by ligation at the splice site of the self-splicing intron places the regulatory elements in position upstream (5′) of the payload sequence such that the regulatory element regulates expression of the payload sequence. In some embodiments, the regulatory element is an IRES. In some embodiments, the regulatory element includes a riboregulator such as a toehold riboregulator. In some embodiment, the regulatory element is a muting sequence

In general, linear RNAs have a shorter half-life than do circular RNAs. Typically, the half-life of linear RNA in cells is regulated at different levels such that mRNAs display a wide range of stability under a given physiologic conditions. Thus, modulation of mRNA stability provides a powerful means for controlling gene expression. Methods for measuring the half-life of mRNA in mammalian cells are known in the art (see e.g., Chen et al. (2008) Methods Enzymol. 2008; 448: 335-357). Accordingly, the linear RNA polynucleotide disclosed herein comprises features that facilitate intracellular circularization e.g., homology arms complementary to target sequences in a target RNA which draw the 3′ and 5′ splice sites of the self-splicing introns or intron fragments, into proximity of each other such that they can ligate, thereby forming circular RNA. The half-life of a circular RNA is extended over what would be possible for a linear RNA. Thus, the administration of the linear RNA polynucleotides disclosed herein, allow for the simultaneous targeting and concentration of therapeutic agents where needed, reducing or avoiding altogether the risk of aberrant expression in off-target tissues and/or cells. Furthermore, because the half-life is extended, the methods disclosed herein may permit less frequent administration thereby further reducing toxicity and improving efficacy.

Thus, the linear RNA polynucleotide circularizes in particular cell/tissue types that express the target RNA by virtue of the 5′ and 3′ homology arms. The homology arms hybridize with target sequences, preferably RNA sequences, specifically produced by the target cell or tissue. Hybridization of the homology arms with the target RNA brings together the 3′ and 5′ splice sites of the self-splicing intron, thereby allowing formation of a circular RNA from which the payload sequence can be expressed. In embodiments, the payload sequence is translated as a therapeutic protein. In embodiments, circularization of the linear RNA polynucleotide places an TRES in position upstream of the payload sequence that is a protein coding sequence, such that the IRES controls translation of the payload sequence. Each element of the linear RNA polynucleotide is discussed in detail below.

### A. Homology Arms

A homology arm is any contiguous sequence that is predicted to form base pairs with at least about 75% (e.g., at least about 80%, at least about 85%, at least about 90%, at least about 95%, about 100%) of a target RNA and which does not hybridize with the other homology arm. Typically, a homology arm is at least 1 nucleotide long and no longer than about 250 nucleotides and located before and adjacent to, or included within, the 3′ intron fragment and/or after and adjacent to, or included within, the 5′ intron fragment and, optionally, is predicted to have less than 50% (e.g., less than 45%, less than 40%, less than 35%, less than 30%, less than 25%) base pairing with unintended sequences in the target RNA. In embodiments, a homology arm has a melting temperature about 35° C. or greater, or about 37° C. or greater, or about 40° C. or greater, or about 45° C. or greater, or about 50° C. or greater, or about 55° C. or greater, or about 60° C. or greater, or about 65° C. or greater, or about 70° C. or greater or about 75° C. or greater or about 80° C. or greater, or about 85° C. or greater when base paired with a complementary region of the target RNA. In preferred embodiments, a homology arm as a melting temperature between about 37° C. and about 65° C. when base paired with a complementary region of the target RNA.

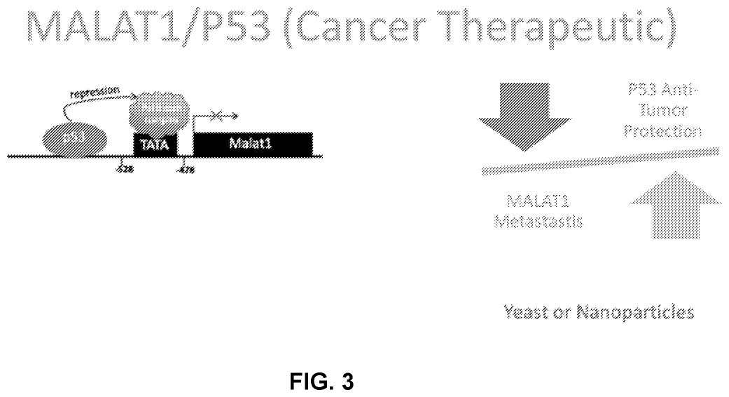

As disclosed herein, the homology arms of the linear RNA polynucleotide play a central role in targeting and circularizing the linear RNA polynucleotide. The homology arms of the linear RNA polynucleotide are complementary to an RNA expressed in a cell or tissue where expression of the payload is desired. For example, as illustrated in FIG. 3, and as described in Example 2, MALAT1, a non-coding RNA specifically expressed in cancer cells, is targeted with a linear RNA polynucleotide having homology arms complementary to a sequence in MALAT1. The linear RNA polynucleotide comprises a payload sequence that encodes the protein p53. Hybridization of the homology arms with MALAT1 brings the splice sites of the 5′ and 3′ self-splicing introns into proximity such that they can join/ligate, thereby forming a circular RNA comprising the coding sequence for the p53 protein payload. Circularization places the TRES in a position upstream of the p53 such that the IRES controls translation of p53. Expression of the p53 is then able to block further expression of MALAT1 (see e.g., FIG. 3).

Typically, the 5′ and 3′ homology arms of the linear RNA polynucleotide comprises a homology region of between about 10 and 250 nucleotides that is complementary to a corresponding target sequence in a tissue or cell specific RNA. In preferred embodiments, the homology arms are each at least 15 nucleotides in length, or at least 10 nucleotides in length. The homology arms are designed such that the 5′ homology arm and the 3′ homology arm each independently bind/hybridize to target sequences in the target RNA. In some embodiments, the homology arms bind/hybridize along their full length with the target RNA. In some embodiments, the duplex formed by hybridization of the homology arms and the target RNA forms a “bubble” at the junction of the 5′ and 3′ homology arms. In some embodiments, the “bubble” can be minimized or eliminated by increasing the strength of hybridization in the vicinity of the junction e.g., by increasing the G-C content. In some embodiments, there is a gap between target sequences in the target RNA such that hybridization of the homology arms is not contiguous along the length of the target sequence. Typically, the gap is 1 nucleotide in length, 2 nucleotides in length, 3 nucleotides, 4 nucleotides, 5 nucleotides, 6 nucleotides, 7 nucleotides, 8 nucleotides, 9 nucleotides, or 10 nucleotides in length. However, in some embodiments, the gap may be greater, for example in a case where the target RNA has a three-dimensional structure whereby non-adjacent sequences along the target RNA strand are positioned proximal to one another in three-dimensional space, for example in a situation where the target RNA is a transfer (tRNA).

The homology regions bind (hybridize) to complementary sequences in the target RNA using Watson-Crick base pairing such that the self-splicing introns on either end of the polynucleotide are drawn into proximity where they can auto-catalytically join, thereby forming a circular RNA. In embodiments, the formation of a circular RNA increases the stability and half-life of the RNA. Additionally, in embodiments, circularization of the linear RNA may bring an IRES and/or other regulatory element located downstream (3′) of the payload sequence in the linear RNA polynucleotide, into position upstream, at the 5′ end, of the payload sequence such that the IRES functions to permit translation of the payload sequence (e.g., encoding a therapeutic protein).

a. Target Sequence

The term “target RNA” as used herein, refers to an RNA expressed primarily or exclusively in one or more target cells or tissues of interest. The sequence of a target RNA targeted by the homology arms is referred to herein as a “target sequence” or a “target RNA sequence.” A “target sequence” is, in embodiments, a contiguous sequence of the “target RNA” that is at least 5 nucleotides long and no longer than 250 nucleotides long, that forms or is predicted to form base pairs with at least about 75% (e.g., at least about 80%, at least about 85%, at least about 90%, at least about 95%, about 100%) of the complementary homology arms of a linear RNA polynucleotide as disclosed herein. Typically, a “target sequence” exhibits less than 50% (e.g., less than 45%, less than 40%, less than 35%, less than 30%, less than 25%, less than 20%, less than 15%, less than 10%, less than 5%, less than 1%) base pairing with unintended sequences in the linear RNA polynucleotide. A “target sequence” can be in a coding or non-coding region of a “target RNA.”

Target sequences may be selected from regions of non-coding sequence, such as e.g., the 3′ untranslated region (UTR) of a target RNA. Thus, in some embodiments, expression/translation of the protein encoded by a target RNA may continue even after being targeted by the homology arms of the linear RNA polynucleotide. However, homology arms may be complementary to any target sequence within a target RNA. In some embodiments, target sequences may comprise the 5′ UTR, or a portion thereof, of a target RNA. In additional or alternative embodiments, target sequences may comprise, or may comprise a portion of, a translation start site of a target RNA. In additional or alternative embodiments, target sequences may comprise, or may comprise a portion of, a coding sequence of a target RNA.

In some embodiments, the linear RNA polynucleotide hybridizes at the 3′ end of a target RNA in target cells. By doing so, the translation of the target RNA, and thereby the expression of its associated protein, can continue unaffected. This can be important, especially in scenarios where the target RNA encodes essential proteins for the normal functioning of the target cell.

In some embodiments, target sequences may be selected to comprise, or comprise a portion of e.g., the 5′ UTR and/or translation start site, to block expression of the target RNA upon recognition of the target sequence by the disclosed homology arms. Such an embodiment may be advantageous in a situation where the target RNA is deleterious, e.g., toxic, or mutated, or encoding an undesired protein such as a defective/non-functional protein, a disease-causing protein, etc. In such an example, the target RNA expression may be blocked, and a desired protein (e.g., therapeutic protein) may be expressed following circularization of the RNA polynucleotide, as herein disclosed.

Such approaches can be used for counteracting harmful effects of certain target RNAs, for example, those encoding mutant proteins that contribute to disease pathogenesis. For example, this approach may be used to selectively downregulate an undesired (e.g., mutated or defective) target RNA, while concurrently expressing a desired version (e.g., a wild type version) of the protein as the payload of the linear RNA polynucleotide. Such a design may be used to replace a defective protein with its functional counterpart, correcting a molecular defect at the heart of a disease in a cell-specific manner.

In embodiments, the linear RNA polynucleotide may be designed to target non-coding target RNAs, which play crucial roles in regulating gene expression. Depending on the function of the non-coding RNA, the linear RNA polynucleotide may either enhance or inhibit its activity. This approach may be used to fine-tune cellular processes and/or rectify aberrant gene regulation associated with various disorders. Thus, by targeting different types of target RNAs or different locations on the target RNAs, the linear RNA polynucleotides described herein can not only disrupt pathological processes but also promote the restoration of normal cellular function.

In embodiments, linear RNA polynucleotides with homology arms for such target RNAs may be used to specifically target cell(s) or tissue(s) expressing a high level of the deleterious RNAs to inhibit the expression of the deleterious RNA and deliver a protein with the restored or correct function in such cell(s) or tissue(s). In one example, a linear RNA polynucleotide with a payload expressing Factor VIII may be designed to target a deficient Factor III mRNA in hepatic cells for treating Hemophilia A. In another example, a linear RNA polynucleotide with a payload expressing Factor IX may be designed to target a deficient Factor IX mRNA in hepatic cells for treating Hemophilia B. In another example, a linear RNA polynucleotide with a payload expressing CFTR may be designed to target a deficient CFTR mRNA in hepatic cells for treating Cystic Fibrosis. In another example, a linear RNA polynucleotide with a payload expressing Micro-dystrophin may be designed to target a deficient Dystrophin mRNA in hepatic cells for treating Duchenne Muscular Dystrophy. In another example, a linear RNA polynucleotide with a payload expressing insulin may be designed to target a deficient insulin mRNA in pancreatic beta cells for treating Type I diabetes. In another example, a linear RNA polynucleotide with a payload expressing Alpha-1 antitrypsin may be designed to target a deficient Alpha-1 antitrypsin mRNA in hepatic cells for treating Alpha-1 Antitrypsin Deficiency.

In embodiments, the selectivity and/or sensitivity of payload delivery in a desired type of cell or tissue may be enhanced by delivering a plurality of linear RNAs, each of which comprises the same payload sequence but comprises different homology arms for binding to a plurality of target RNAs primarily or exclusively expressed in the desired type of cell or tissue. In such cases, the expression of the payload may be highly selective in the selected cell or tissue.

As such, even in cases where one or more of the target RNAs is expressed at a low level, the payload can still be expressed at a high level with this approach, thus also enhancing the sensitivity of payload delivery to the selected cell or tissue. For example, the expression of a target RNA may not be exclusively limited to a specific type of cell or tissue, but it may also be found at lower levels in some healthy tissues. To refine the cell-type specificity of the linear RNA activation and to minimize potential off-target effects, the linear RNA polynucleotides may be used to target multiple target RNAs, each of which may be expressed at lower levels individually but taken together are collectively expressed at a higher therapeutic level in the desired target diseased tissue than in healthy cells.

Hybridization of target sequences with the homology arms is driven by Watson-Crick base pairing. Hybridization of the homology arms with the complementary target sequences draws the self-splicing introns or intron fragments into close proximity where they can auto-catalytically link to one another at their respective splice sites to form a circular RNA.

b. Melting Temperature of Duplexes Formed Between Homology Arms and Target Sequences

Hybridization between the homology arms and the complementary target sequences must be sufficiently strong and stable to draw the self-splicing introns or intron fragments into a proximity where they are can auto-catalytically link to one another at their respective splice sites to form a circular RNA. Accordingly, the homology arms are designed and the complementary target sites are identified such that the duplexes formed by pairings between the two have sufficient strength to carry out the circularization function.

Melting temperature of a duplex nucleic acid is a measure of the strength of hybridization between the molecules that comprise the duplex. Accordingly, in designing homology arms and selecting target sequences, the melting temperature of the duplexes formed are evaluated. Methods for determining the melting temperature of a duplex nucleic acid are well known in the art see e.g., Dumousseau, M., et al. (2012) supra; Wetmur J (1991) DNA probes: applications of the principles of nucleic acid hybridization. Crit Rev Biochem Mol Biol 26:227-259; Huang F, et al. (2009) Partition function and base pairing probabilities for RNA-RNA interaction prediction. Bioinformatics 25(20):2646-2654; Owczarzy R, et al. (2008) Predicting stability of DNA duplexes in solutions containing Magnesium and Monovalent Cations. Biochemistry 47:5336-5353; Tan Z J, et al. (2006) Nucleic acid helix stability: effects of Salt concentration, cation valence and size, and chain length. Bioph J 90:1175-1190. In some embodiments, the Tm of duplexes formed between a homology arm and a target sequence are optimized to promote circularization of the liner RNA polynucleotide in a target cell.

Typically, the melting temperature of a homology arm/target sequence duplex is in a range that is between about 35° C. and about 60° C., although higher melting temperatures are contemplated herein. Typically, the longer the payload sequence, the stronger the duplex needs to be to stabilize the binding/hybridization and thereby facilitate circularization. In some embodiments where the linear RNA polynucleotide comprises a muting sequence as herein disclosed, the muting sequence may have a greater affinity for a complementary portion of a self-splicing intron of the linear RNA polynucleotide when the homology arm(s) are not paired with the target RNA, and a lesser affinity for the complementary portion of the self-splicing intron when homology arm(s) are paired with the target sequences of the target RNA. In this way, upon the homology arm(s) encountering the target sequence(s), the binding thereto may disrupt the binding of the muting sequence with the complementary sequence of the linear RNA polynucleotide, thereby relieving the inhibitory aspect of the muting sequence on RNA circularization.

### B. Self-Splicing Introns

a. Group I Introns

Group I introns are structured self-splicing introns that in part persist in genomes by minimizing the impact of their insertion into host genes. Group I introns are known in the art see e.g., Hausner et al. (2014) Mobile DNA 5:8; Clancy, S. (2008) RNA splicing: introns, exons and spliceosome. Nature Education 1(1):31; Hedberg, A., Johansen, S. D. (2013) Nuclear group I introns in self-splicing and beyond. Mobile DNA 4: 17). Group I introns all fold into a complex secondary structure with nine loops and employ transesterification reactions as described below.

Two transesterifications characterize the mechanism by which group I introns are spliced: 3′OH of a free guanine nucleoside (or one located in the intron) or a nucleotide cofactor (GMP, GDP, GTP) attacks phosphate at the 5′ splice site. 3′OH of the 5′ exon becomes a nucleophile and the second transesterification results in the joining of the two exons. As discussed below, inverting the location of the 3′ and 5′ intron splice sites by comparison to the wild type group I introns, permits formation of a circular RNA.

Examples of Group I intron self-splicing sequences include, but are not limited to, self-splicing permuted intron-exon sequences derived from T4 bacteriophage gene td, Cyanobacterium Anabaena sp. pre-tRNA-Leu gene, Tetrahymena thermophila group I intron, Candida group I intron, or Azoarcus group I intron. Therefore, in some embodiments, the 3′ group I intron fragment and 5′ group I intron fragment are Anabaena group I intron fragments.

b. Group II Introns

Group II introns are structured self-splicing introns see e.g., Lambowitz et al. Cold Spring Harb Perspect Biol 2011; 3:a003616; Clancy (2008), supra. Group II introns are ribozymes that catalyze their own splicing via two sequential transesterification reactions. Similar to group I introns, group II introns are self-splicing in that they form a ribozyme and are capable of splicing without the assistance of any proteins. However, the specific splicing mechanism differs from that of the group I introns.

In particular, the mechanism by which group II introns are spliced (two transesterification reaction like group I introns) is as follows: The 2′OH of a specific adenosine in the intron attacks the 5′ splice site, thereby forming the lariat. The 3′OH of the 5′ exon triggers the second transesterification at the 3′ splice site, thereby joining the exons together. As discussed below, inverting the location of the 3′ and 5′ intron splice sites by comparison to the wild type group II introns, permits formation of a circular RNA.

c. Permuted Intron-Exon (PIE) Sequences

“Permutated intron-exon” or “PIE” sequences refers to a self-splicing intron configuration wherein the location of the 3′ and 5′ intron splice site sequences on a linear RNA polynucleotide is inverted compared to wild type, such that the 3′ end of the exon is joined to a splice site at an upstream rather than a downstream position (donor (5′) splice site is 3′ of the acceptor (3′) splice site) thereby permitting formation of a circular RNA. Using self-splicing permuted intron-exon (PIE) sequences in which the order of the splice sites has been reversed, it is possible to catalyze the circularization of a variety of RNA exon sequences in vitro, as well as in vivo in bacteria and in yeast (Puttaraju, M. and Been, (1992) supra, and GenBank X69005). Circular RNAs generated by splicing have been demonstrated with in vitro manipulated Group I and Group II intron sequences. Permuted intron-exon (PIE) sequences are known in the art (see e.g., Puttaraju & Been, (1992) Nuc. Acids Res. 20:5357-5364; Petkovic, S. & Muller, S., RNA circularization strategies in vivo and in vitro, Nucleic Acids Research, 43(4):2454-2465 (2015)).

d. Hammerhead Ribozymes

In some embodiments, the linear RNA polynucleotide comprises a hammerhead ribozyme as the self-splicing intron. The hammerhead ribozyme can facilitate the circularization of the linear RNA polynucleotide. In some examples, the 5′ and 3′ ends of the linear RNA polynucleotide may comprise sequences forming part of the hammerhead ribozyme structure, e.g., with one component of the hammerhead ribozyme on the 5′ end and the other on the 3′ end. Upon hybridization of the homology arms to a target RNA, the two components of the hammerhead ribozyme structure may come into close proximity, forming a functional ribozyme. Such ribozyme has the ability to undergo self-cleavage and ligation reactions.

The self-cleavage reaction involves a nucleophilic attack by the 2′ oxygen of a specific ribose on the adjacent phosphodiester bond, which leads to the breakage of the bond and the formation of a 2′,3′-cyclic phosphate end and a 5′-hydroxyl end. Following the self-cleavage, the ligation step occurs. The 2′,3′-cyclic phosphate end may react with the 5′-hydroxyl end in a transesterification reaction, resulting in the formation of a 3′-5′ phosphodiester bond, thereby circularizing the RNA molecule. This circularization process may be dependent on the presence of the target RNA to bring the hammerhead ribozyme components into close proximity. By designing the sequences adjacent to the hammerhead ribozyme components to be complementary to the target RNA, the circularization of the linear RNA polynucleotide may be made cell-type specific. The relatively small sizes of hammerhead ribozymes may make the design and synthesis of linear RNA polynucleotides easier and more flexible.

### C. Payload Sequences

In some embodiments, the payload sequence encodes a polypeptide or peptide of interest, e.g. for therapeutic or diagnostic purposes.

a. Therapeutic Proteins

In embodiments, the linear RNA polynucleotide comprises a payload sequence for expressing a therapeutic protein. Accordingly, in embodiments, the disclosure provides a linear RNA polynucleotide comprising, the following elements operably-linked to each other and arranged in the in the following order: (i) a 5′ homology arm, (ii) a 3′ self-splicing intron fragment containing a 3′ splice site dinucleotide, (iii) optionally, a 5′ spacer sequence, (iv) a payload sequence, (v) an IRES (vi) optionally, a 3′ spacer sequence, (vii) a 5′ self-splicing intron fragment containing a 5′ splice site dinucleotide, and (viii) a 3′ homology arm, wherein the 5′ homology arm and the 3′ homology arm are complementary to target sequences in a target RNA and are not complementary to each other; and wherein binding/hybridization of the homology arms to the target RNA allows production of a circular RNA in a cell or tissue comprising the target sequence. Circularization of the linear RNA polynucleotide places the TRES in position upstream (5′) of the protein coding region where it can control translation of the protein coding region.

In embodiments, in the linear RNA polynucleotide, the IRES is positioned 3′ to the payload sequence. In this location, the IRES is non-functional with respect to promoting translation of the payload sequence. Circularization of the linear RNA polynucleotide relocates the IRES to a position 5′ (upstream) of the payload sequence thereby positioning it to exert translational control. Cell specific expression is ensured because translation of the RNA cannot occur unless the RNA is first circularized. Circularization is cell-specific by virtue of the homology arms which hybridize with a desired target sequence in a target RNA.

The payload sequence can encode any protein of eukaryotic or prokaryotic origin, or a fragment thereof. Therapeutic proteins as herein disclosed can include but are not limited to insulin, pramlintide acetate, growth hormone, pegvisoman, mecasermin, factor VIII, factor IX, protein C concentrate, α1-proeinase inhibitor, erythropoietin, filgrastim. Exemplary therapeutic proteins relevant to the present disclosure are described in Dimitrov, DS, Methods Mol Biol. 899: 1-26 (2012), the contents of which is incorporated herein by reference in its entirety. Exemplary therapeutic proteins can include, e.g., an antibody or a functional fragment thereof. In some embodiments, the therapeutic protein is an anti-viral antibody. In exemplary embodiments, the therapeutic protein comprises a SARS-CoV-2 spike protein nanobody. In other embodiments, the therapeutic protein is a monoclonal antibody, e.g. an anti-COVID-19 M protein antibody or an anti-COVID-19 NC protein antibody. In some embodiments, the therapeutic protein comprises a C. difficile SLP nanobody. In some embodiments, the therapeutic protein is an anti-inflammatory antibody, e.g., an anti-TNFa monoclonal antibody. In an exemplary embodiment, the therapeutic protein comprises the heavy and light chains from adalimumab (HUMIRA®). In embodiments, the therapeutic protein is an anti-cancer antibody, e.g., a Herceptin antibody for breast cancer.

In embodiments, the payload encodes an antibody or a fragment thereof that binds to an antigen, e.g., CD19, CD123, CD22, CD30, CD171, CS-1, C-type lectin-like molecule-1, CD33, epidermal growth factor receptor variant III (EGFRvIII), disialoganglioside GD2, disaloganglioside GD3, TNF receptor family member, B cell maturation antigen (BCMA), Tn antigen ((Tn Ag) or (GalNAca-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, HER2, HER3, Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), claudin 18.2 (CLDN18.2), thyroid stimulating hormone receptor (TSHR), G protein-coupled receptor class C group 5, member D (GPRC5D), chromosome X open reading frame 61 (CXORF61), CD97, or CD179a.

In embodiments, the therapeutic protein is a hormone (e.g. insulin, ghrelin, leptin, and the like), an enzyme (e.g. alcohol dehydrogenase or other detoxifying enzymes), a cytokine (e.g. IL-10), an anti-microbial protein (Iseganan (IB-367) or hLF1-11) a chemokine, a mitogen, an immunogen, (e.g. Covid S protein or fragment thereof), a growth factor (e.g. human growth hormone), or a differentiation factor (OSK, Yamanaka factors for tissue regeneration).

In embodiments, the payload sequence encodes a regulatory protein, e.g., tumor protein p53 (TP53), which acts as a tumor suppressor. In other embodiments, the payload sequence encodes Serapina1, a serine protease inhibitor.

In embodiments, the payload sequence encodes a protein with tumor suppression activity, and the linear RNA polynucleotide with such payload sequence may be used for treating cancers. For example, such payload sequence may encode Tumor Necrosis Factor-related Apoptosis-inducing Ligand (TRAIL), Herpes Simplex Virus Thymidine Kinase (HSV-TK), P53, Interleukin-12 (IL-12), Interferon-gamma (IFN-gamma), Pro-drug enzymes (e.g., cytosine deaminase (CD) and nitroreductase (NTR)), Suicide enzymes (e.g., cytosine deaminase), GM-CSF (Granulocyte-Macrophage Colony-Stimulating Factor), Caspase-8, or Apoptin.

Linear RNA polynucleotides comprising such payload sequences may have homology arms for targeting cancer-specific or cancer-associated RNAs. Examples of cancer-specific or cancer-associated target RNAs include Prostate-Specific Antigen (PSA) mRNA (e.g., for treating prostate cancer), Human Epidermal growth factor Receptor 2 (HER2) mRNA (e.g., for treating breast and gastric cancers), B-cell lymphoma 2 (BCL-2) mRNA e.g., for treating various types of cancers including leukemia, lymphoma, and melanoma), Epidermal Growth Factor Receptor (EGFR) mRNA (e.g., for treating non-small-cell lung carcinoma, glioblastoma, and other cancers), Telomerase Reverse Transcriptase (TERT) mRNA (e.g., for treating multiple cancer types), Survivin (BIRC5) mRNA (for treating many types of cancers including lung and breast cancer), C-Myc mRNA (e.g., for treating Burkitt's lymphoma and other cancers), CD19 mRNA (e.g., for treating B-cell malignancies), Melanoma Antigen Family (MAGE) mRNA (e.g., for treating various types of cancers including melanoma, breast cancer, and non-small cell lung cancers).

Having the benefit of the present disclosure and invention, the skilled artisan can readily ascertain a wide range of suitable combinations of payloads and homology arms recognizing target RNAs, such that the linear RNA polynucleotides may be used for treating various diseases. Examples of combinations are shown in Table 1 below.

TABLE 1

| Payload | Target RNA | Indication |
|---|---|---|
| Herpes simplex virus | Prostate-specific antigen | Prostate |
| thymidine kinase (HSV- | (PSA) mRNA | Cancer |
| TK) | | |
| Apoptin | Human epidermal growth | Breast Cancer |
| | factor receptor 2 (HER2) | |
| | mRNA | |
| Interleukin-12 (IL-12) | Melanocyte protein (Pmel) | Melanoma |
| | mRNA | |
| Tumor necrosis factor- | Mucin 1 (MUC1) mRNA | Pancreatic |
| related apoptosis- | | Cancer |
| inducing ligand (TRAIL) | | |
| CASP8 | Doublecortin (DCX) | Neuroblastoma |
| | mRNA | |

In embodiments, the payload comprises a sequence encoding a therapeutic protein for treating diabetes. In one example, the therapeutic protein is glucagon-like peptide (GLP-1). In another example, the therapeutic protein is insulin. In cases where the therapeutic protein is insulin, the linear RNA polynucleotide may comprise 5′ and 3′ homology arms binding to a target RNA that is specifically expressed in pancreatic p cells, the natural site of insulin production, thus mimicking the body's natural insulin production. In the cases where the therapeutic protein is GLP-1, the linear RNA polynucleotide may comprise 5′ and 3′ homology arms binding to a target RNA that is specifically expressed in intestinal L cells, the natural site of GLP-1 production, thus mimicking the body's natural GLP-1 production.

In embodiments, the linear RNA polynucleotides are designed for treating autoimmune diseases. For example, the linear RNA polynucleotides may be designed or treating type 1 diabetes. In type 1 diabetes, an autoimmune response leads to the destruction of P islet cells in the pancreas, causing insulin deficiency and hyperglycemia. Traditional therapeutic approaches, such as insulin replacement, manage the symptoms but do not address the underlying loss of 3 cells. However, the specificity and versatility of the linear RNA polynucleotides disclosed herein can be used to deliver therapeutic proteins such as survival factors and/or stem cell differentiation signals specifically to pancreatic p cells, thus slowing pancreatic 3 cell death and/or regenerating lost cells.

In embodiments, the linear RNA polynucleotide may comprise 5′ and 3′ homology arms binding to a target RNA specifically expressed in pancreatic R cells (e.g., insulin), and a payload sequence encoding a differentiation factor. The differentiation factors may induce the transformation of pancreatic progenitor cells or other suitable cell types into insulin-producing p cells, e.g., replenishing the lost population. Examples of differentiation factors include Pancreatic and Duodenal Homeobox 1 (PDX1), Neurogenin 3 (NGN3), V-Maf Musculoaponeurotic Fibrosarcoma Oncogene Family, Protein A (MAFA), NK6 Homeobox 1 (NKX6-1), Paired Box 6 (PAX6), GLP-1, Betacellulin (BTC), Insulin-Like Growth Factor 1 (IGF-1), Epidermal Growth Factor (EGF), and Hepatocyte Growth Factor (HGF).

In embodiments, the linear RNA polynucleotide may comprise 5′ and 3′ homology arms binding to a target RNA specifically expressed in pancreatic R cells (e.g., insulin), and a payload encoding a survival factor. The survival factor may slow the rate of pancreatic R cell death, e.g., providing a larger window of time for intervention in early-stage type 1 diabetes. Examples of survival factors include Insulin-Like Growth Factor 1 (IGF-1), Glucose-dependent insulinotropic polypeptide (GIP), Glucagon-Like Peptide 1 (GLP-1), Epidermal Growth Factor (EGF), Platelet-Derived Growth Factor (PDGF), Exendin-4, Galectin-3, Hepatocyte Growth Factor (HGF), Transforming Growth Factor alpha (TGF-α), and Connective Tissue Growth Factor (CTGF).

Additional examples of suitable proteins or peptides that can be advantageously encoded by the payload sequence in the subject invention include those described in US2023/0048732 (e.g., paragraph [0111] and Table 1), US2023/0058784 (e.g., paragraphs [0298]-[0410]), and US2022/0117902 (e.g., paragraphs [0184]-[0191]), each of which is expressly incorporated by reference herein in its entirety.

In embodiments, the protein or peptide encoded by the payload may comprise a cleavage site. For example, the cleavage site may be a furin cleavage site, e.g., the sites described in Oh S et al., GLP-1 Gene Delivery for the Treatment of Type 2 Diabetes, Molecular Therapy, Vol. 7, No. 4, April 2003; Groskreutz D J et al., Genetically Engineered Proinsulin Constitutively Processed and Secreted as Mature, Active Insulin, JBC, Vol. 269, No. 8, Issue of February 25, pp. 6241-6245, 1994, each of which is incorporated by reference in its entirety.

In embodiments, the payload sequence may encode multiple proteins, peptides, or domains, with protease cleavage sites in between. In such cases, the payload may be translated into a fusion protein comprising the multiple proteins, peptides, or domains, and the fusion protein may be further processed by one or more proteases that cleaves at the cleavage sites to produce the desired plurality of proteins. In some examples, the cleavage site is a 2A peptide cleavage site, which is a short self-cleaving peptide able to mediate cleavage of peptides during translation in cells, e.g., as described in WO 2020/223478, which is incorporated by reference in its entirety.

In embodiments, the payload sequence may encode a reporter protein or peptide that can be visualized or detected in cells (e.g., when expressed, the reporter protein or peptide emits fluorescence or luminescence). In such cases, the expression of the payload may be indicative of the presence of a target RNA. Such linear RNA polynucleotide may be used in diagnostics, e.g., for detecting the presence of a disease-associated RNA, in vivo or in vitro. Examples of the payloads include coding sequences for luciferase and fluorescent proteins such as green fluorescent protein, blue fluorescent protein, cyan fluorescent protein, red fluorescent protein, yellow fluorescent protein or any photoconvertible protein. The expression of the reporter protein or peptide may be readily detected by imaging techniques well known in the art. For example, the expression of the reporter protein or peptide may be detected by whole-body imaging techniques, such as bioluminescence imaging (BLI) for luciferase or fluorescence imaging (e.g., GFP). Such approaches may be used to monitor the expression of the target RNA(s) in live organisms and/or in real-time. In embodiments, these linear RNAs may be used to monitor the activity of specific genes in patients or animal models during disease progression or in response to therapeutic interventions, and/or to trace cellular differentiation pathways in developmental biology studies.

The payload sequence may further comprise elements for proper cellular localization of the protein, e.g., nuclear localization sequence (NLS) (e.g., SV40 NLS). Examples of NLS sequences include those described in US20190093107A1 (e.g., paragraph [0231]), which is incorporated by reference herein in its entirety.

b. IRES Elements

Expression of heterologous genes in eukaryotic cells is important for gene transfer and gene therapy protocols. As is known in the art, translation of eukaryotic genes can be achieved by using the internal ribosome binding site (IRES) element to initiate translation (see e.g., Al-Allaf et al., (2019) Non-coding RNA Research 4(1):1-14). Internal ribosome entry site (IRES) elements are cis-acting RNA regions that promote internal initiation of protein synthesis using cap-independent mechanisms. Accordingly, in embodiments, expression of the protein coding region of a linear RNA polynucleotide is controlled at least in part by at least one IRES.

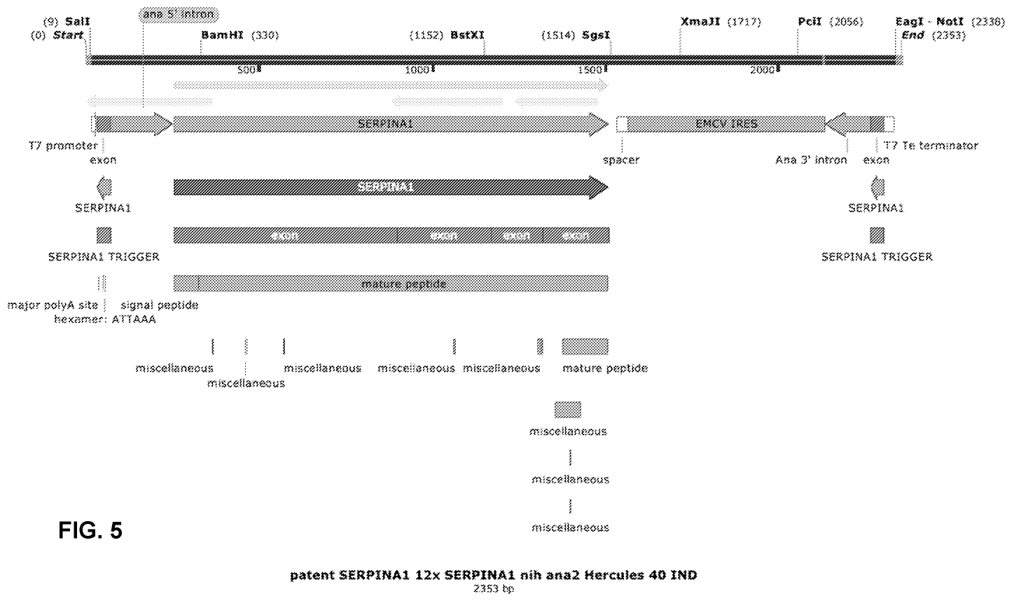

According to some embodiments of the subject invention, in the linear RNA polynucleotide, the IRES is located 3′ (downstream) to the payload sequence see e.g., FIG. 5. As such, the IRES cannot direct translation of the payload sequence either in vitro, or in a non-targeted cell. However, upon circularization of the RNA polynucleotide in an appropriately targeted cell, the IRES is brought into position 5′ to the payload sequence where it can direct translation of the payload.

Accordingly, in embodiments, the linear RNA polynucleotides provided herein may comprise at least one IRES. Inclusion of an IRES permits the translation of one or more open reading frames from a circular RNA (e.g., open reading frames that form the expression sequence) when operably linked and located 5′ to a payload sequence. The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20: 102-110; Kobayashi et al., BioTechniques (1996) 21:399-402; and Mosser et al., BioTechniques 1997 22 150-161).

A multitude of TRES sequences are available and include sequences derived from a wide variety of viruses, such as from leader sequences of picornaviruses such as the encephalomyocarditis virus (EMCV) UTR (Fang et al. J. Virol. (1989) 63: 1651-1660), the polio leader sequence, the hepatitis A virus leader, the hepatitis C virus IRES, human rhinovirus type 2 IRES (Dobrikova et al., Proc. Natl. Acad. Sci. (2003) 100(25): 15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279(5):3389-3397), and the like.

In some embodiments, the IRES is an IRES sequence of Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SHI, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Salivirus A BNS, Salivirus A BN2, Salivirus A 02394, Salivirus A GUT, Salivirus A CH, Salivirus A SZ1, Salivirus FHB, CVB3, CVB1, Echovirus 7, CVBS, EVA71, CVA3, CVA12, EV24 or an aptamer to eIF4G. In some embodiments, the IRES is an TRES sequence of EMCV. In some embodiments, the IRES is an IRES sequence of CVB3. Further examples of IRES include the IRES sequences in the database IRESite: The database of experimentally verified IRES structures (www.iresite.org/). For example, the IRES may be the IRES of any one of AQP4, BCL2, c-IAP1_-1178/-64, CVB3, ELG1, EMCV, HCV_type_1a, HRV-2, Kv1.4, LamB1_-335_-1, LEF1, Pim-1, UtrA, XIAP, CrPV, CSFV, HIV, BiP, or HvB3.

In some embodiments, a riboregulator or riboswitch may be included in a linear RNA polynucleotide such that IRES function is inhibited until an inhibitory aspect of the riboregulator or riboswitch is relieved. Riboregulators and riboswitches are known in the art (see e.g., Krishnamurthy (2015) supra, Edwards, A. L. & Batey, R. T. Cold Spring Harb Perspect Biol 2011; 3:a003533). An exemplary riboregulator is a “toehold riboregulator,” disclosed e.g., in U.S. Pat. Nos. 9,550,987 and 11,124,846, the disclosures of which are expressly incorporated by reference herein.

For example, in embodiments a riboregulator or riboswitch is employed in the linear RNA polynucleotide such that the IRES element adopts a conformation whereby translation of the payload sequence is prevented. Upon the riboregulator recognizing another sequence in a cell, or riboswitch recognizing a particular small molecule, the recognition may induce a conformational change that relieves the inhibitory aspect, thereby permitting translation of the payload sequence. Thus, translation of the payload sequence can be prevented in a cell/tissue type that does not include an RNA sequence recognized by the riboregulator, or de-repressive small molecule that functions to relieve the inhibitory nature of the riboswitch, on expression of a therapeutic protein.

Accordingly, in further embodiments, expression of a payload (e.g., a therapeutic protein) may be prevented following circularization of the linear RNA polynucleotide as herein disclosed, unless the circularized RNA is in a cell/tissue type where the inhibitory nature of the riboregulator or riboswitch with regard to IRES function can also be relieved. This may advantageously allow for a second level of therapeutic protein expression control. For example, it is herein recognized that in some examples there may be some level of background circularization of a linear RNA polynucleotide. By including a riboregulator or riboswitch as herein disclosed that functions to disrupt IRES function in cells lacking a corresponding RNA molecule or other small molecule capable to relieve the inhibitory aspect, expression of the therapeutic protein will be prevented in cell/tissue types that do not also express said RNA molecule or other small molecule. In this way, specificity of the cell/tissue type where the therapeutic protein is expressed may be improved. In some embodiments herein, the riboregulator is a toehold riboregulator.

Via the use of a riboregulator or riboswitch that disrupt IRES functional aspect(s), it is also herein recognized that, advantageously, more than one IRES sequence can be included in a linear RNA polynucleotide, whereby the payload (e.g., the therapeutic protein) expression occurs only when the inhibitory aspect of the particular IRES function is relieved. Via strategic riboregulator/riboswitch selection, such a strategy may enable temporal and spatial control of the expression of more than one therapeutic protein.

In embodiments, one or more muting sequences as herein disclosed may also be included in a linear RNA polynucleotide sequence to inhibit circularization of the linear RNA polynucleotide until, e.g., the target RNA is recognized via the homology arm(s). Examples of muting sequences include those described in Ying Y. et al., Nucleic Acid Nanotechnology for Diagnostics and Therapeutics in Acute Kidney Injury, Int J Mol Sci. 2022 Mar. 13; 23(6):3093, which is incorporated by reference herein in its entirety. This may further serve to improve cell/tissue-type selectivity by preventing/reducing circularization of the linear RNA polynucleotide in cell/tissue types lacking the target RNA. In some examples, the muting sequence may be downstream of the 3′ homology arm on the linear RNA polynucleotide. In some examples, the muting sequence may be upstream of the 5′ homology arm on the linear RNA polynucleotide.

Thus, in some embodiments expression of payload(s) (e.g., therapeutic protein(s)) in particular cell/tissue type(s) may be subject to multiple levels of control. As a representative example, a linear RNA polynucleotide may be designed such that a muting sequence prevents circularization in the absence of a target RNA that is recognized by homology arm(s) of the linear RNA polynucleotide. Once circularized, a second level of control may comprise a riboregulator or riboswitch that prevents expression of the payload (e.g., therapeutic protein) unless the inhibitory nature of the riboregulator or riboswitch can also be de-repressed via the expression of a particular RNA in the case of a riboregulator (which in some examples could be the target RNA, or another RNA molecule), or presence of small molecule in the case of a riboswitch. Thus, in an example where a riboregulator is used to selectively repress translation of the payload sequence and where a muting sequence is used to selectively repress RNA circularization, only cells that express, for example, both the target RNA and a second RNA will receive the payload (e.g., therapeutic protein). Specifically, in this example, recognition of the target RNA by the homology arm(s) relieves the inhibitory aspect of the muting sequence and enables RNA circularization. Then, once circularized, expression of the payload (e.g., therapeutic protein) is inhibited via the riboregulator unless a second RNA is expressed (or a small molecule is present) that is recognized by the riboregulator (or riboswitch). Once the riboregulator recognizes the second RNA, expression of the payload (e.g., therapeutic protein) may occur.

Other similar examples are within the scope of this disclosure. Such control may be advantageous for diseases/conditions in which more than one gene is involved, such that the payload(s) (e.g., therapeutic protein(s)) are delivered specifically to those cells that exhibit a particular RNA profile.

### D. Spacer Sequences

“Structured” RNA refers to an RNA sequence that is predicted by the RNAFold software or similar predictive tools to form a structure (e.g., a hairpin loop) with itself or other sequences in the same RNA molecule. Highly structured RNA such as is found e.g., in IRES and/or introns, may interfere with the folding of the splicing ribozyme, thereby reducing the efficiency of circularization. Therefore, in order to improve circularization efficiency, in some embodiments, the linear RNA polynucleotide comprises “spacer sequences.” The spacer sequences allow the structured nucleic acid sequences structures to fold independently, thereby improving the efficiency of circular RNA generation. The use of “spacer sequences” is known in the art see e.g., U.S. Pat. No. 11,352,640.

Thus, in some embodiments, the linear RNA polynucleotide comprises a 5′ and/or a 3′ spacer sequence. A “spacer” refers to any contiguous nucleotide sequence that occurs between elements of the linear RNA polynucleotide and which is included to reduce or avoid the interference between proximal structures. For example, to reduce or avoid the interference between the folding of different structures in the linear RNA polynucleotide a spacer sequence may be inserted between the elements. Therefore, in some embodiments, the linear RNA polynucleotide comprises a spacer sequence between an IRES element and the self-splicing intron-derived sequences. Typically a “spacer” is about 7 nucleotides long (and optionally no longer than 100 nucleotides) and is located downstream of and adjacent to the 3′ intron fragment and/or upstream of and adjacent to the 5′ intron fragment.

In one embodiment, the linear RNA polynucleotide comprises a 5′ spacer sequence, but not a 3′ spacer sequence. In another embodiment, the linear RNA polynucleotide comprises a 3′ spacer sequence, but not a 5′ spacer sequence. In another embodiment, the linear RNA polynucleotide comprises neither a 5′ spacer sequence, nor a 3′ spacer sequence. In one embodiment, the linear RNA polynucleotide comprises both a 5′ spacer sequence and a 3′ spacer sequence.

Typically, a “spacer” comprises one or more of the following: a) an unstructured region at least 5 nucleotides long, b) a region of predicted base pairing at least 5 nucleotides long to a distal (i.e., non-adjacent) sequence, including another spacer, and/or c) a structured region at least 1 nucleotide long limited in scope to the sequence of the spacer.

In embodiments, the spacer sequence can be, for example, at least 10 nucleotides in length, at least 15 nucleotides in length, or at least 30 nucleotides in length. In some embodiments, the spacer sequence is at least 7, 8, 9, 10, 11, 12, 13, 14, 15, 16, 17, 18, 19, 20, 25 or 30 nucleotides in length. In embodiments, the spacer sequence is no more than 100, 90, 80, 70, 60, 50, 45, 40, 35 or 30 nucleotides in length. In embodiments the spacer sequence is between 20 and 50 nucleotides in length. In embodiments, the spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length.

The spacer sequences can be polyA sequences, polyA-C sequences, polyC sequences, or poly-U sequences, or the spacer sequences can be specifically engineered depending on the IRES. Spacer sequences as disclosed herein can have two functions: (1) promote circularization and (2) promote functionality by allowing the introns and IRES to fold correctly. Thus, the spacer sequences as disclosed herein are designed 1) to be inert with regards to the folding of proximal intron and IRES structures; 2) to sufficiently separate intron and IRES secondary structures; and 3) to contain a region of spacer-spacer complementarity to promote the formation of a “splicing bubble.”

### II. RNA Synthesis

A linear RNA polynucleotide can be prepared using any method known in the art. For example, a linear RNA polynucleotide may be prepared using recombinant DNA methodology well known in the art (see Sambrook et al., 2001, Molecular Cloning: A Laboratory Manual, Cold Spring Harbor Laboratory Press, New York; Ausubel et al., 2001, Current Protocols in Molecular Biology, Green & Wiley, New York) or for example, by direct synthesis. For recombinant and in vitro transcription, a DNA construct encoding the linear RNA polynucleotide can be obtained from known clones, by synthesizing a DNA molecule encoding an RNA molecule, or by cloning the elements comprising the RNA polynucleotide. Techniques for in vitro transcription of RNA molecules and methods for cloning genes encoding known RNA molecules are described by, for example, Sambrook et al, supra.

Thus, in some embodiments, the disclosure provides a DNA construct for synthesizing a linear RNA polynucleotide disclosed herein. Typically a DNA construct for preparing a linear RNA polynucleotide as disclosed herein comprises at least the following elements arranged in the following sequence (i) a transcriptional promoter; (ii) a 5′ target homology arm; (iii) a 3′ self-splicing group I intron fragment comprising a 3′ splice site dinucleotide; (iv) optionally, a 5′ spacer sequence; (v) a payload sequence; (vi) an internal ribosome entry site (IRES); (vii) optionally, a 3′ spacer sequence; (viii) a 5′ self-splicing group I intron fragment comprising a 5′ splice site dinucleotide; and (ix) a 3′ target homology arm, wherein the 5′ homology arm and the 3′ homology are not complementary to each other.

Methods for synthesizing nucleic acids de novo typically involve the organic synthesis of a nucleic acid from nucleoside derivatives. This synthesis may be performed in solution or on a solid support. One type of organic synthesis is the phosphotriester method, which has been used to prepare gene fragments or short genes. In the phosphotriester method, oligonucleotides are prepared which can then be joined together to form longer nucleic acids. For a description of this method, see Narang, et al., (1979, Meth. Enzymol., 68: 90) and U.S. Pat. No. 4,356,270. The phosphotriester method can be used in the present invention to synthesize an isolated RNA.

In addition, the compositions of the present invention can be synthesized in whole or in part, or an isolated RNA can be conjugated to another nucleic acid using organic synthesis such as the phosphodiester method, which has been used to prepare a tRNA gene. See Brown, et al. (1979, Meth. Enzymol., 68: 109) for a description of this method. As in the phosphotriester method, the phosphodiester method involves synthesis of oligonucleotides which are subsequently joined together to form the desired nucleic acid.

A third method for synthesizing nucleic acids, described in U.S. Pat. No. 4,293,652, is a hybrid of the above-described organic synthesis and molecular cloning methods. In this process, the appropriate number of oligonucleotides to make up the desired nucleic acid sequence is organically synthesized and inserted sequentially into a vector which is amplified by growth prior to each succeeding insertion.

In addition, molecular biological methods, such as using a nucleic acid as a template for a PCR, or cloning appropriate nucleic acid elements into a vector and transforming a cell with the vector can be used to make large amounts of the linear RNA polynucleotide of the present invention.

The linear RNA polynucleotides disclosed herein may be optimized to improve its function (e.g., expression) by known approaches in the art. For example, the untranslated regions, IRES, and the expression vector topology may be optimized as described in Chen et al., Engineering circular RNA for enhanced protein production, Nat. Biotechnol. 2023 February; 41(2):262-272, which is incorporated by reference herein in its entirety.

### III. Delivery Systems

The linear RNA polynucleotide disclosed herein can be delivered to cells and tissues using any method known in the art. In embodiments, the linear RNA polynucleotide provided herein may be delivered and/or targeted to a cell in a delivery vehicle, e.g., a liposome, a nanoparticle, or a composition comprising a nanoparticle. In some embodiments, the delivery vehicle is a nanoparticle. In some embodiments, the nanoparticle is a lipid nanoparticle, a non-lipid polymeric core-shell nanoparticle, or a biodegradable nanoparticle. In some embodiments, the delivery vehicle comprises one or more polymers, cationic lipids, non-cationic lipids, ionizable lipids, PEG-modified lipids, polyglutamic acid lipids, Hyaluronic acid lipids, poly β-amino esters, poly beta amino peptides, or positively charged peptides.

In one embodiment, the delivery vehicle is selected and/or prepared to optimize delivery of the linear RNA to a target cell. For example, if the target cell is a hepatocyte the properties of the delivery vehicle (e.g., size, charge and/or pH) may be optimized to effectively deliver such delivery vehicle to the target cell, reduce immune clearance and/or promote retention in that target cell. Alternatively, if the target cell is the central nervous system (e.g., linear RNA administered for the treatment of neurodegenerative diseases may specifically target brain or spinal tissue), selection and preparation of the delivery vehicle must consider penetration of; and retention within the blood brain barrier and/or the use of alternate means of directly delivering such transfer vehicle to such target cell. In one embodiment, the compositions disclosed herein are combined with agents that facilitate the transfer of exogenous RNA (e.g., agents that disrupt or improve the permeability of the blood brain barrier and thereby enhance the transfer of exogenous linear RNA to the target cells).

a. Lipid Nanoparticles

In some embodiments, the delivery vehicle is polymer-based. Suitable polymers may include, for example; polyorganic amines (e.g., polyethyleneimine (PEI), polyethyleneimine celluloses, and derivatives thereof); poly(amidoamines) (PAMAM and derivatives thereof); polyamino acids (e.g., polylysine (PLL), polyarginine, and derivatives thereof); polysaccharides (e.g., cellulose, dextran, DEAE dextran, starch); polyacrylates, polyalkycyanoacrylates, polylactide, polylactide-polyglycolide copolymers, polycaprolactones, dextran, albumin, gelatin, alginate, collagen, chitosan, cyclodextrins, dendrimers; spermine, spermidine, poly(vinylbenzyl trialkyl ammonium), poly(4-vinyl-N-alkyl-pyridiumiun), poly(acryloyl-trialkyl ammonium), and Tat proteins. See, e.g., Samal et al., Cationic polymers and their therapeutic potential, Chem Soc Rev. 41:7147-94 (2012). In one embodiment, the delivery vehicle is selected based upon its ability to facilitate the transfection of a linear RNA to a target cell.

In some embodiments, the delivery vehicle is lipid-based. Examples of suitable lipids include the phosphatidyl compounds (e.g., phosphatidylglycerol, phosphatidylcholine, phosphatidylserine, phosphatidylethanolamine, sphingolipids, cerebrosides, and gangliosides); esters of phosphatidic acid with an aminoalcohol, such as an ester of dipalmitoyl phosphatidic acid or distearoyl phosphatidic acid with hydroxyethylenediamine. More particular examples of positively charged lipids include 3β-[N—(N′,N′-dimethylaminoethyl)carbamoyl) cholesterol (DC-chol); N,N′-dimethyl-N,N′-dioctacyl ammonium bromide (DDAB); N,N′-dimethyl-N,N′-dioctacyl ammonium chloride (DDAC); 1,2-dioleoyloxypropyl-3-dimethyl-hydroxyethyl ammonium chloride (DORI), 1,2-dioleoyloxy-3-[trimethylammonio]-propane (DOTAP); N-(1-(2,3-dioleyloxy)propyl)-N,N,N-trimethylammonium chloride (DOTMA); dipalmitoylphosphatidylcholine (DPPC); 1,2-dioctadecyloxy-3-[trimethylammonio]-propane (DSTAP); and the cationic lipids described in e.g. Martin et al., Current Pharmaceutical Design 2005, 11, 375-394.

Blends of lipids and polymers in any concentration and in any ratio can also be used. Blending different polymer types in different ratios using various grades can result in characteristics that borrow from each of the contributing polymers. Various terminal group chemistries can also be adopted.

The disclosure contemplates the use of lipid nanoparticles as delivery vehicles comprising a cationic lipid to encapsulate and/or enhance the delivery of linear RNA into the target cell that will act as a depot for protein production. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available.

Suitable cationic lipids for use in the compositions and methods disclosed herein include those described e.g., in international patent application publication WO 2010/053572 or Felgner et al. (Proc. Nat'l Acad. Sci. 84, 7413 (1987); U.S. Pat. No. 4,897,355; WO 2010/042877; Semple et al., Nature Biotech. 28:172-176 (2010), Heyes, J., et al., J Controlled Release 107: 276-287 (2005); Morrissey, D V., et al., Nat. Biotechnol. 23(8): 1003-1007 (2005); or international patent publication WO2005/121348A1.

The use of cholesterol-based cationic lipids is also contemplated by the present disclosure. Such cholesterol-based cationic lipids can be used, either alone or in combination with other cationic or non-cationic lipids. Suitable cholesterol-based cationic lipids are disclosed for example, in Gao, et al. Biochem. Biophys. Res. Comm. 179, 280 (1991); Wolf et al. BioTechniques 23, 139 (1997); U.S. Pat. No. 5,744,335.

In addition, several reagents are commercially available to enhance transfection efficacy. Suitable examples include LIPOFECTIN (DOTMA:DOPE) (Invitrogen, Carlsbad, Calif), LIPOFECTAMINE (DOSPA:DOPE) (Invitrogen), LIPOFECTAMINE2000. (Invitrogen), FUGENE (Promega, Madison, Wis.), TRANSFECTAM (DOGS) (Promega), and EFFECTENE (Qiagen, Valencia, Calif).

Also contemplated are cationic lipids such as the dialkylamino-based, imidazole-based, and guanidinium-based lipids, such as those described in U.S. Pat. No. 10,413,618.

The use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000](C8 PEG-2000 ceramide) is may also be used, either alone or in combination with other lipids together which comprise the delivery vehicle (e.g., a lipid nanoparticle). Contemplated PEG-modified lipids include, but are not limited to, a polyethylene glycol chain of up to 5 kDa in length covalently attached to a lipid with alkyl chain(s) of C6-C20 length. The addition of such components may prevent complex aggregation and may also provide a means for increasing circulation lifetime and increasing the delivery of the lipid-nucleic acid composition to the target cell, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivatized lipids of the present disclosure may comprise a molar ratio from about 0% to about 20%, about 0.5% to about 20%, about 1% to about 15%, about 4% to about 10%, or about 2% of the total lipid present in the delivery vehicle.

The present disclosure also contemplates the use of non-cationic lipids. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. Such non-cationic lipids may be used alone or in combination with other excipients, for example, cationic lipids. When used in combination with a cationic lipid, the non-cationic lipid may comprise a molar ratio of 5% to about 90%, or about 10% to about 70% of the total lipid present in the delivery vehicle.

The delivery vehicle (e.g., a lipid nanoparticle) may be prepared by combining multiple lipid and/or polymer components. For example, a delivery vehicle may be prepared using C12-200, DOPE, cholesterol, DMG-PEG2K at a molar ratio of 40:30:25:5, or DODAP, DOPE, cholesterol, DMG-PEG2K at a molar ratio of 18:56:20:6, or HGT5000, DOPE, cholesterol, DMG-PEG2K at a molar ratio of 40:20:35:5, or HGT5001, DOPE, cholesterol, DMG-PEG2K at a molar ratio of 40:20:35:5. The selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the linear RNA to be delivered. Additional considerations include, for example, the saturation of the alkyl chain, as well as the size, charge, pH, pKa, fusogenicity and toxicity of the selected lipid(s). Thus, the molar ratios may be adjusted accordingly. For example, in some embodiments, the percentage of cationic lipid in the lipid nanoparticle may be greater than 10%, greater than 20%, greater than 30%, greater than 40%, greater than 50%, greater than 60%, or greater than 70%. The percentage of non-cationic lipid in the lipid nanoparticle may be greater than 5%, greater than 10%, greater than 20%, greater than 30%, or greater than 40%. The percentage of cholesterol in the lipid nanoparticle may be greater than 10%, greater than 20%, greater than 30%, or greater than 40%. The percentage of PEG-modified lipid in the lipid nanoparticle may be greater than 1%, greater than 2%, greater than 5%, greater than 10%, or greater than 20%.

The delivery vehicles for use in the compositions of the disclosure can be prepared by various techniques which are presently known in the art. Multi-lamellar vesicles (MLV) may be prepared using conventional techniques, for example, by depositing a selected lipid on the inside wall of a suitable container or vessel, dissolving the lipid in an appropriate solvent, and then evaporating the solvent to leave a thin film on the inside of the vessel or by spray-drying. An aqueous phase may then be added to the vessel with a vortexing motion which results in the formation of MLVs. Uni-lamellar vesicles (ULV) can then be formed by homogenization, sonication or extrusion of the multi-lamellar vesicles. In addition, ULV can be formed by detergent removal techniques.

In certain embodiments of this disclosure, the compositions disclosed herein comprise a delivery vehicle wherein the linear RNA is associated on both the surface of the delivery vehicle and encapsulated within the same delivery vehicle. For example, during preparation of the compositions of the disclosed herein, cationic delivery vehicles may associate with the linear RNA through electrostatic interactions.

b. Yeast Based Delivery Vehicles

In embodiments, the linear RNA polynucleotide disclosed herein can be delivered orally as a yeast based composition. Yeast based oral compositions are disclosed e.g., in co-pending international application PCT/US2021/060865, filed Nov. 24, 2021, which is incorporated herein by reference in its entirety. To facilitate delivery using a yeast based delivery system, the linear RNA polynucleotide is modified to further comprise a nucleic acid binding peptide ligand sequence that facilitates packaging by the yeast based system. In an exemplary embodiment, the nucleic acid binding peptide ligand sequence is an MS2 ligand sequence.

c. Carbon Nanoparticles

In embodiments, the linear RNA polynucleotide is captured on a carbon nanoparticle as disclosed e.g., in U.S. Pat. No. 10,344,274. The captured nucleic acid and carbon particle is subsequently combined with lipofectamine or other lipid-based structures and delivered to cells by standard methods see e.g., Mendes et al. (2022) Nanodelivery of nucleic acids. Nat Rev Methods Primers 2, 24 (2022).

### IV. Pharmaceutical Formulations

The present invention also provides “pharmaceutically acceptable” or “physiologically acceptable” formulations. Such formulations can be administered in vivo to a subject in order to practice treatment methods.

As used herein, the terms “pharmaceutically acceptable” and “physiologically acceptable” refer to carriers, diluents, excipients and the like that can be administered to a subject, preferably without producing excessive adverse side-effects (e.g., nausea, abdominal pain, headaches, etc.). Such preparations for administration include sterile aqueous or non-aqueous solutions, suspensions, and emulsions. Liquid formulations include suspensions, solutions, syrups and elixirs. Liquid formulations may be prepared by the reconstitution of a solid.

Pharmaceutical formulations can be made from carriers, diluents, excipients, solvents, dispersion media, coatings, antibacterial and antifungal agents, isotonic and absorption delaying agents, and the like, compatible with administration to a subject. Such formulations can be contained in a tablet (coated or uncoated), capsule (hard or soft), microbead, emulsion, powder, granule, crystal, suspension, syrup or elixir. Supplementary active compounds and preservatives, among other additives, may also be present, for example, antimicrobials, anti-oxidants, chelating agents, and inert gases and the like.

Excipients can include a salt, an isotonic agent, a serum protein, a buffer or other pH-controlling agent, an anti-oxidant, a thickener, an uncharged polymer, a preservative or a cryoprotectant. Excipients used in compositions of the invention may further include an isotonic agent and a buffer or other pH-controlling agent. These excipients may be added for the attainment of preferred ranges of pH (about 6.0-8.0) and osmolarity (about 50-400 mmol/L). Examples of suitable buffers are acetate, borate, carbonate, citrate, phosphate and sulfonated organic molecule buffer. Such buffers may be present in a composition in concentrations from 0.01 to 1.0% (w/v). An isotonic agent may be selected from any of those known in the art, e.g. mannitol, dextrose, glucose and sodium chloride, or other electrolytes. Preferably, the isotonic agent is glucose or sodium chloride. The isotonic agents may be used in amounts that impart to the composition the same or a similar osmotic pressure as that of the biological environment into which it is introduced. The concentration of isotonic agent in the composition will depend upon the nature of the particular isotonic agent used and may range from about 0.1 to 10%. When glucose is used, it is preferably used in a concentration of from 1 to 5% w/v, more particularly 5% w/v. When the isotonic agent is sodium chloride, it is preferably employed in amounts of up to 1% w/v, in particular 0.9% w/v. The compositions of the invention may further contain a preservative. Examples preservatives are polyhexamethylene-biguanidine, benzalkonium chloride, stabilized oxychloro complexes (such as those known as Purite®), phenylmercuric acetate, chlorobutanol, sorbic acid, chlorhexidine, benzyl alcohol, parabens, and thimerosal. Typically, such preservatives are present at concentrations from about 0.001 to 1.0%. Furthermore, the compositions of the invention may also contain a cryopreservative agent. Preferred cryopreservatives are glucose, sucrose, mannitol, lactose, trehalose, sorbitol, colloidal silicon dioxide, dextran of molecular weight preferable below 100,000 g/mol, glycerol, and polyethylene glycols of molecular weights below 100,000 g/mol or mixtures thereof. Most preferred are glucose, trehalose and polyethylene glycol. Typically, such cryopreservatives are present at concentrations from about 0.01 to 10%.

A pharmaceutical formulation can be formulated to be compatible with its intended route of administration. For example, for oral administration, a composition can be incorporated with excipients and used in the form of tablets, troches, capsules, e.g., gelatin capsules, or coatings, e.g., enteric coatings (Eudragit® or Sureteric®). Pharmaceutically compatible binding agents, and/or adjuvant materials can be included in oral formulations. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or other stearates; a glidant such as colloidal silicon dioxide; a sweetening agent such as sucrose or saccharin; or a flavoring agent such as peppermint, methyl salicylate, or flavoring.

Formulations can also include carriers to protect the composition against rapid degradation or elimination from the body, such as a controlled release formulation, including implants and microencapsulated delivery systems. For example, a time delay material such as glyceryl monostearate or glyceryl stearate alone, or in combination with a wax, may be employed.

Suppositories and other rectally administrable formulations (e.g., those administrable by enema) are also contemplated. Further regarding rectal delivery, see, for example, Song et al., Mucosal drug delivery: membranes, methodologies, and applications, Crit. Rev. Ther. Drug. Carrier Syst., 21:195-256, 2004; Wearley, Recent progress in protein and peptide delivery by noninvasive routes, Crit. Rev. Ther. Drug. Carrier Syst., 8:331-394, 1991.

Additional pharmaceutical formulations appropriate for administration are known in the art and are applicable in the methods and compositions of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; and Pharmaceutical Principles of Solid Dosage Forms, Technonic Publishing Co., Inc., Lancaster, Pa., (1993)).

Delivery systems useful in the context of embodiments of the invention may also include time-released, delayed release, and sustained release delivery systems such that the delivery of the compositions occurs prior to, and with sufficient time to cause, sensitization of the site to be treated. The composition can be used in conjunction with other therapeutic agents or therapies. Such systems can avoid repeated administrations of the composition, thereby increasing convenience to the subject and the physician, and may be particularly suitable for certain composition embodiments of the invention.

Release delivery systems include polymer base systems such as poly(lactide-glycolide), copolyoxalates, polyesteramides, polyorthoesters, polycaprolactones, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; sylastic systems; peptide based systems; hydrogel release systems; wax coatings; compressed tablets using conventional binders and excipients; partially fused implants; and the like. In some embodiments, lipid nanoparticles or polymers are used as delivery vehicles for therapeutic circRNAs described herein, including delivery of RNA to tissues.

In embodiments, the administration of the compositions may be carried out in any manner, e.g., by parenteral or nonparenteral administration, including by aerosol inhalation, injection, infusions, ingestion, transfusion, implantation or transplantation. For example, the compositions described herein may be administered to a patient trans-arterially, intradermally, subcutaneously, intratumorally, intramedullary, intranodally, intramuscularly, by intravenous (i.v.) injection, intranasally, intrathecally or intraperitoneally. In one aspect, the compositions of the present disclosure are administered intravenously. In one aspect, the compositions of the present disclosure are administered to a subject by intradermal or subcutaneous injection. The compositions may be injected, for instance, directly into a tumor, lymph node, tissue, organ, or site of infection.

### Examples

Example 1: The following Example illustrates the nucleic acid sequence corresponding to the DNA construct shown in FIG. 1.

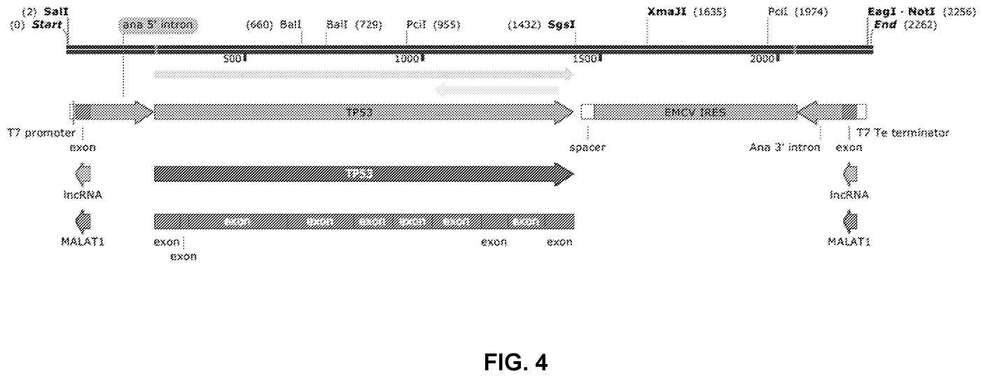

Example 2: The following Example illustrates an exemplary DNA construct that encodes a linear RNA polynucleotide for cell specific targeting of cancer. FIG. 3 illustrates regulation of metastasis-associated lung adenocarcinoma transcript 1 (MALAT1). MALAT1 is up-regulated in human cancer, and has been shown to induce cancer cell proliferation, migration, and invasion in vitro, and tumor metastasis in mice. Thus, repression of MALAT1 by p53 as illustrated schematically in FIG. 3 increases anti-tumor protection and decreases metastasis promoted by MALAT1. Accordingly, the DNA sequence shown in SEQ ID NO: 1 encodes a linear RNA encoding a p53 protein targeted to the 3′ UTR of MALAT1 by its homology arms as disclosed herein. Successful targeting of MALAT1 transcripts brings about circularization of the linear RNA encoding the p53 and places the IRES 5′ to the p53 coding sequence to permit translation of the p53 protein. Translation of the p53 protein in turn represses expression of MALAT1. The DNA constructs, linear RNA, protein products produced on circularization of the linear RNA, and overall experiment is illustrated graphically in FIG. 4.

SEQ ID NO: 1: Malat1p53
GGGAACACAACTTTTAATCATACTCCAGTCGTTTCACAATAACAATAGATGACTTACAACTAAT
CGGAAGGTGCAGAGACTCGACGGGAGCTACCCTAACGTCAAGACGAGGGTAAAGAGAGAGTCCA
ATTCTCAAAGCCAATAGGCAGTAGCGAAAGCTGCAAGAGAATGAAAATCCGTTGACCTTAAACG
GTCGTGTGGGTTCAAGTCCCTCCACCCCCAATGGAGGAGCCGCAGTCAGATCCTAGCGTCGAGC
CCCCTCTGAGTCAGGAAACATTTTCAGACCTATGGAAACTACTTCCTGAAAACAACGTTCTGTC
CCCCTTGCCGTCCCAAGCAATGGATGATTTGATGCTGTCCCCGGACGATATTGAACAATGGTTC
ACTGAAGACCCAGGTCCAGATGAAGCTCCCAGAATGCCAGAGGCTGCTCCCCCCGTGGCCCCTG
CACCAGCAGCTCCTACACCGGCGGCCCCTGCACCAGCCCCCTCCTGGCCCCTGTCATCTTCTGT
CCCTTCCCAGAAAACCTACCAGGGCAGCTACGGTTTCCGTCTGGGCTTCTTGCATTCTGGGACA
GCCAAGTCTGTGACTTGCACGTACTCCCCTGCCCTCAACAAGATGTTTTGCCAACTGGCCAAGA
CCTGCCCTGTGCAGCTGTGGGTTGATTCCACACCCCCGCCCGGCACCCGCGTCCGCGCCATGGC
CATCTACAAGCAGTCACAGCACATGACGGAGGTTGTGAGGCGCTGCCCCCACCATGAGCGCTGC
TCAGATAGCGATGGTCTGGCCCCTCCTCAGCATCTTATCCGAGTGGAAGGAAATTTGCGTGTGG
AGTATTTGGATGACAGAAACACTTTTCGACATAGTGTGGTGGTGCCCTATGAGCCGCCTGAGGT
TGGCTCTGACTGTACCACCATCCACTACAACTACATGTGTAACAGTTCCTGCATGGGCGGCATG
AACCGGAGGCCCATCCTCACCATCATCACACTGGAAGACTCCAGTGGTAATCTACTGGGACGGA
ACAGCTTTGAGGTGCGTGTTTGTGCCTGTCCTGGGAGAGACCGGCGCACAGAGGAAGAGAATCT
CCGCAAGAAAGGGGAGCCTCACCACGAGCTGCCCCCAGGGAGCACTAAGCGAGCACTGCCCAAC
AACACCAGCTCCTCTCCCCAGCCAAAGAAGAAACCACTGGATGGAGAATATTTCACCCTTCAGA
TCCGTGGGCGTGAGCGCTTCGAGATGTTCCGAGAGCTGAATGAGGCCTTGGAACTCAAGGATGC
CCAGGCTGGGAAGGAGCCAGGGGGGAGCAGGGCTCACTCCAGCCACCTGAAGTCCAAAAAGGGT
CAGTCTACCTCCCGCCATAAAAAACTCATGTTCAAGACAGAAGGGCCTGACTCAGACTGAGGCG
CGCCTTGCCTAGAAGGCCGGAAACCAACTTTATTACTATATTCCCCACAACCCCCCTCTCCCTC
CCCCCCTAACGTTACTGGCCGAAGCCGCTTGGAATAAGGCCGGTGTGCGTTTGTCTATATGTTA
TTTTCCACCATATTGCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTGTTGAATGTCGTGAA
GGAAGCAGTTCCTCTGGAAGCTTCTTGAAGACAAACAACGTCTGTAGCGACCCTTTGCAGGCAG
CGGAACCCCCCACCTGGCGACAGGTGCCTCTGCGGCCAAAAGCCACGTGTATAAGATACACCTG
CAAAGGCGGCACAACCCCAGTGCCACGTTGTGAGTTGGATAGTTGTGGAAAGAGTCAAATGGCT
CTCCTCAAGCGTATTCAACAAGGGGCTGAAGGATGCCCAGAAGGTACCCCATTGTATGGGATCT
GATCTGGGGCCTCGGTGCACATGCTTTACATGTGTTTAGTCGAGGTTAAAAAACGTCTAGGCCC
CCCGAACCACGGGGACGTGGTTTTCCTTTGAAAAACACGATGATAATAGACGCTACGGACTTAA
ATAATTGAGCCTTAAAGAAGAAATTCTTTAAGTGGATGCTCTCAAACTCAGGGAAACCTAAATC
TAGTTATAGACAAGGCAATCCTGAGCCAAGCCGAAGTAGTAATTAGTAAGCATTCTAATAGCAG
CGGGATCAGAACAGTACTGCATTTA

The Nucleotide positions of the components in the construct are shown in Table 2 below.

TABLE 2

| Components | Nucleotides |
|---|---|
| 5′ homology arm | 27-66 |
| 3′ self-splicing intron | 67-248 |
| Payload | 249-1430 |
| spacer | 1454-1486 |
| IRES | 1487-2057 |
| 5′ self-splicing intron | 2058-2187 |
| 3′ homology arm | 2188-2227 |

Example 3: The following Example illustrates the DNA sequence of an exemplary construct for producing a linear RNA encoding Serapina 1, for use in Liver Specific protein replacement using defective Serpina1 as target RNA, and correct Alpha-1 Antitrypsin (Serpina1) as cargo. The DNA constructs, linear RNA, and protein products produced on circularization of the linear RNA are illustrated graphically in FIG. 5.

SEQ ID NO: 2: Serapina 1
GGGACCAGCTCAACCCTTCTTTAATGTCATCCAGGGAGGGAACAATAGATGACTTACAACTAAT
CGGAAGGTGCAGAGACTCGACGGGAGCTACCCTAACGTCAAGACGAGGGTAAAGAGAGAGTCCA
ATTCTCAAAGCCAATAGGCAGTAGCGAAAGCTGCAAGAGAATGAAAATCCGTTGACCTTAAACG
GTCGTGTGGGTTCAAGTCCCTCCACCCCCAATGCCGTCTTCTGTCTCGTGGGGCATCCTCCTGC
TGGCAGGCCTGTGCTGCCTGGTCCCTGTCTCCCTGGCTGAGGATCCCCAGGGAGATGCTGCCCA
GAAGACAGATACATCCCACCATGATCAGGATCACCCAACCTTCAACAAGATCACCCCCAACCTG
GCTGAGTTCGCCTTCAGCCTATACCGCCAGCTGGCACACCAGTCCAACAGCACCAATATCTTCT
TCTCCCCAGTGAGCATCGCTACAGCCTTTGCAATGCTCTCCCTGGGGACCAAGGCTGACACTCA
CGATGAAATCCTGGAGGGCCTGAATTTCAACCTCACGGAGATTCCGGAGGCTCAGATCCATGAA
GGCTTCCAGGAACTCCTCCGTACCCTCAACCAGCCAGACAGCCAGCTCCAGCTGACCACCGGCA
ATGGCCTGTTCCTCAGCGAGGGCCTGAAGCTAGTGGATAAGTTTTTGGAGGATGTTAAAAAGTT
GTACCACTCAGAAGCCTTCACTGTCAACTTCGGGGACACCGAAGAGGCCAAGAAACAGATCAAC
GATTACGTGGAGAAGGGTACTCAAGGGAAAATTGTGGATTTGGTCAAGGAGCTTGACAGAGACA
CAGTTTTTGCTCTGGTGAATTACATCTTCTTTAAAGGCAAATGGGAGAGACCCTTTGAAGTCAA
GGACACCGAGGAAGAGGACTTCCACGTGGACCAGGTGACCACCGTGAAGGTGCCTATGATGAAG
CGTTTAGGCATGTTTAACATCCAGCACTGTAAGAAGCTGTCCAGCTGGGTGCTGCTGATGAAAT
ACCTGGGCAATGCCACCGCCATCTTCTTCCTGCCTGATGAGGGGAAACTACAGCACCTGGAAAA
TGAACTCACCCACGATATCATCACCAAGTTCCTGGAAAATGAAGACAGAAGGTCTGCCAGCTTA
CATTTACCCAAACTGTCCATTACTGGAACCTATGATCTGAAGAGCGTCCTGGGTCAACTGGGCA
TCACTAAGGTCTTCAGCAATGGGGCTGACCTCTCCGGGGTCACAGAGGAGGCACCCCTGAAGCT
CTCCAAGGCCGTGCATAAGGCTGTGCTGACCATCGACGAGAAAGGGACTGAAGCTGCTGGGGCC
ATGTTTTTAGAGGCCATACCCATGTCTATCCCCCCCGAGGTCAAGTTCAACAAACCCTTTGTCT
TCTTAATGATTGAACAAAATACCAAGTCTCCCCTCTTCATGGGAAAAGTGGTGAATCCCACCCA
AAAATAAGGCGCGCCTTGCCTAGAAGGCCGGAAACCAACTTTATTACTATATTCCCCACAACCC
CCCTCTCCCTCCCCCCCTAACGTTACTGGCCGAAGCCGCTTGGAATAAGGCCGGTGTGCGTTTG
TCTATATGTTATTTTCCACCATATTGCCGTCTTTTGGCAATGTGAGGGCCCGGAAACCTGGCCC
TGTCTTCTTGACGAGCATTCCTAGGGGTCTTTCCCCTCTCGCCAAAGGAATGCAAGGTCTGTTG
AATGTCGTGAAGGAAGCAGTTCCTCTGGAAGCTTCTTGAAGACAAACAACGTCTGTAGCGACCC
TTTGCAGGCAGCGGAACCCCCCACCTGGCGACAGGTGCCTCTGCGGCCAAAAGCCACGTGTATA
AGATACACCTGCAAAGGCGGCACAACCCCAGTGCCACGTTGTGAGTTGGATAGTTGTGGAAAGA
GTCAAATGGCTCTCCTCAAGCGTATTCAACAAGGGGCTGAAGGATGCCCAGAAGGTACCCCATT
GTATGGGATCTGATCTGGGGCCTCGGTGCACATGCTTTACATGTGTTTAGTCGAGGTTAAAAAA
CGTCTAGGCCCCCCGAACCACGGGGACGTGGTTTTCCTTTGAAAAACACGATGATAATAGACGC
TACGGACTTAAATAATTGAGCCTTAAAGAAGAAATTCTTTAAGTGGATGCTCTCAAACTCAGGG
AAACCTAAATCTAGTTATAGACAAGGCAATCCTGAGCCAAGCCGAAGTAGTAATTAGTAAGGCC
AGGGATGGAGGGGAGGGGTTGAGGAGCGAGAGGCAG

The nucleotide positions of the components in the construct are shown in Table 3 below.

TABLE 3

| Components | Nucleotides |
|---|---|
| 5′ homology arm | 34-73 |
| 3′ self-splicing intron | 74-255 |
| Payload | 256-1512 |
| spacer | 1536-1568 |
| IRES | 1569-2139 |
| 5′ self-splicing intron | 2140-2269 |
| 3′ homology arm | 2270-2309 |

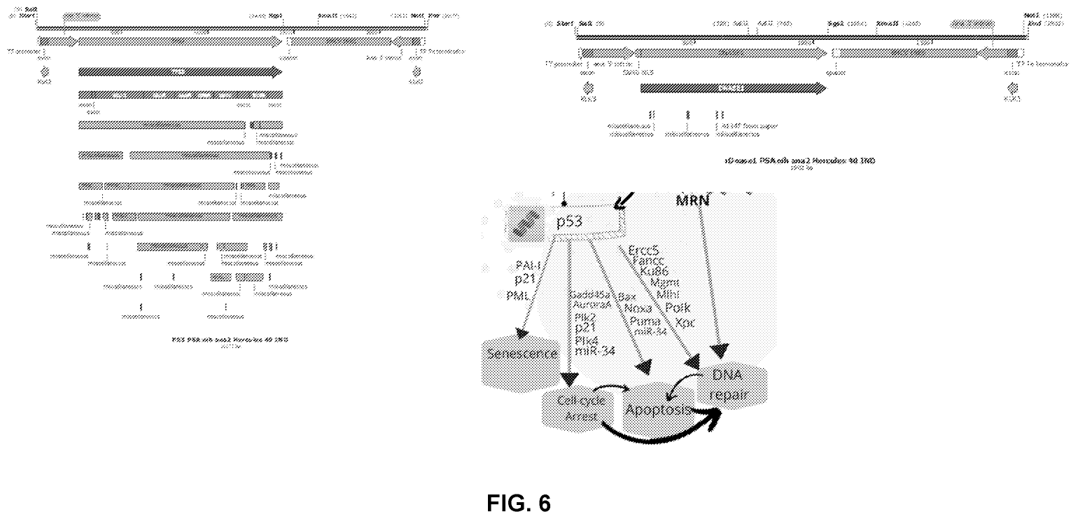

Example 4: The following Example illustrates the DNA sequence of two exemplary DNA constructs for Prostate Specific Apoptosis Induction of Cancer cells using PSA as target RNA. The DNA constructs, linear RNA, protein products produced on circularization of the linear RNA, and overall experiments are illustrated graphically in FIG. 6.

SEQ ID NO: 3: PSA P53
TGGGACAAGGGCCAGGACCATCTGCAGGATGCTGTCCCCGAACAATAGATGACTTACAACTAAT
CGGAAGGTGCAGAGACTCGACGGGAGCTACCCTAACGTCAAGACGAGGGTAAAGAGAGAGTCCA
ATTCTCAAAGCCAATAGGCAGTAGCGAAAGCTGCAAGAGAATGAAAATCCGTTGACCTTAAACG
GTCGTGTGGGTTCAAGTCCCTCCACCCCCAATGGAGGAGCCGCAGTCAGATCCTAGCGTCGAGC
CCCCTCTGAGTCAGGAAACATTTTCAGACCTATGGAAACTACTTCCTGAAAACAACGTTCTGTC
CCCCTTGCCGTCCCAAGCAATGGATGATTTGATGCTGTCCCCGGACGATATTGAACAATGGTTC
ACTGAAGACCCAGGTCCAGATGAAGCTCCCAGAATGCCAGAGGCTGCTCCCCCCGTGGCCCCTG
CACCAGCAGCTCCTACACCGGCGGCCCCTGCACCAGCCCCCTCCTGGCCCCTGTCATCTTCTGT
CCCTTCCCAGAAAACCTACCAGGGCAGCTACGGTTTCCGTCTGGGCTTCTTGCATTCTGGGACA
GCCAAGTCTGTGACTTGCACGTACTCCCCTGCCCTCAACAAGATGTTTTGCCAACTGGCCAAGA
CCTGCCCTGTGCAGCTGTGGGTTGATTCCACACCCCCGCCCGGCACCCGCGTCCGCGCCATGGC
CATCTACAAGCAGTCACAGCACATGACGGAGGTTGTGAGGCGCTGCCCCCACCATGAGCGCTGC
TCAGATAGCGATGGTCTGGCCCCTCCTCAGCATCTTATCCGAGTGGAAGGAAATTTGCGTGTGG
AGTATTTGGATGACAGAAACACTTTTCGACATAGTGTGGTGGTGCCCTATGAGCCGCCTGAGGT
TGGCTCTGACTGTACCACCATCCACTACAACTACATGTGTAACAGTTCCTGCATGGGCGGCATG
AACCGGAGGCCCATCCTCACCATCATCACACTGGAAGACTCCAGTGGTAATCTACTGGGACGGA
ACAGCTTTGAGGTGCGTGTTTGTGCCTGTCCTGGGAGAGACCGGCGCACAGAGGAAGAGAATCT
CCGCAAGAAAGGGGAGCCTCACCACGAGCTGCCCCCAGGGAGCACTAAGCGAGCACTGCCCAAC
AACACCAGCTCCTCTCCCCAGCCAAAGAAGAAACCACTGGATGGAGAATATTTCACCCTTCAGA
TCCGTGGGCGTGAGCGCTTCGAGATGTTCCGAGAGCTGAATGAGGCCTTGGAACTCAAGGATGC
CCAGGCTGGGAAGGAGCCAGGGGGGAGCAGGGCTCACTCCAGCCACCTGAAGTCCAAAAAGGGT
CAGTCTACCTCCCGCCATAAAAAACTCATGTTCAAGACAGAAGGGCCTGACTCAGACTGAGGCG
CGCCTTGCCTAGAAGGCCGGAAACCAACTTTATTACTATATTCCCCACAACCCCCCTCTCCCTC
CCCCCCTAACGTTACTGGCCGAAGCCGCTTGGAATAAGGCCGGTGTGCGTTTGTCTATATGTTA
TTTTCCACCATATTGCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTGTTGAATGTCGTGAA
GGAAGCAGTTCCTCTGGAAGCTTCTTGAAGACAAACAACGTCTGTAGCGACCCTTTGCAGGCAG
CGGAACCCCCCACCTGGCGACAGGTGCCTCTGCGGCCAAAAGCCACGTGTATAAGATACACCTG
CAAAGGCGGCACAACCCCAGTGCCACGTTGTGAGTTGGATAGTTGTGGAAAGAGTCAAATGGCT
CTCCTCAAGCGTATTCAACAAGGGGCTGAAGGATGCCCAGAAGGTACCCCATTGTATGGGATCT
GATCTGGGGCCTCGGTGCACATGCTTTACATGTGTTTAGTCGAGGTTAAAAAACGTCTAGGCCC
CCCGAACCACGGGGACGTGGTTTTCCTTTGAAAAACACGATGATAATAGACGCTACGGACTTAA
ATAATTGAGCCTTAAAGAAGAAATTCTTTAAGTGGATGCTCTCAAACTCAGGGAAACCTAAATC
TAGTTATAGACAAGGCAATCCTGAGCCAAGCCGAAGTAGTAATTAGTAATGGCTCCTCACAGAG
GCCACCTTCCAGGACTACTCTCTGA

The nucleotide positions of the components in the construct are shown in Table 4 below.

TABLE 4

| Components | Nucleotides |
|---|---|
| 5′ homology arm | 34-73 |
| 3′ self-splicing intron | 74-255 |
| Payload | 256-1437 |
| spacer | 1461-1443 |
| IRES | 1444-2064 |
| 5′ self-splicing intron | 2065-2194 |
| 3′ homology arm | 2195-2234 |

SEQ ID NO: 4 PSA RDNAse1
TGGGACAAGGGCCAGGACCATCTGCAGGATGCTGTCCCCGAACAATAGATGACTTACAACTAAT
CGGAAGGTGCAGAGACTCGACGGGAGCTACCCTAACGTCAAGACGAGGGTAAAGAGAGAGTCCA
ATTCTCAAAGCCAATAGGCAGTAGCGAAAGCTGCAAGAGAATGAAAATCCGTTGACCTTAAACG
GTCGTGTGGGTTCAAGTCCCTCCACCCCCACCCCCCAAGAAGAAGAGAAAGGTGCTGAAGATCG
CAGCCTTCAACATCCAGACATTTGGGGAGACCAAGATGTCCAATGCCACCCTCGTCAGCTACAT
TGTGCAGATCCTGAGCCGCTATGACATCGCCCTGGTCCAGGAGGTCAGAGACAGCCACCTGACT
GCCGTGGGGAAGCTGCTGGACAACCTCAATCAGGATGCACCAGACACCTATCACTACGTGGTCA
GTGAGCCACTGGGACGGAACAGCTATAAGGAGCGCTACCTGTTCGTGTACAGGCCTGACCAGGT
GTCTGCGGTGGACAGCTACTACTACGATGATGGCTGCGAGCCCTGCGGGAACGACACCTTCAAC
CGAGAGCCATTCATTGTCAGGTTCTTCTCCCGGTTCACAGAGGTCAGGGAGTTTGCCATTGTTC
CCCTGCATGCGGCCCCGGGGGACGCAGTAGCCGAGATCGACGCTCTCTATGACGTCTACCTGGA
TGTCCAAGAGAAATGGGGCTTGGAGGACGTCATGTTGATGGGCGACTTCAATGCGGGCTGCAGC
TATGTGAGACCCTCCCAGTGGTCATCCATCCGCCTGTGGACAAGCCCCACCTTCCAGTGGCTGA
TCCCCGACAGCGCTGACACCACAGCTACACCCACGCACTGTGCCTATGACAGGATCGTGGTTGC
AGGGATGCTGCTCCGAGGCGCCGTTGTTCCCGACTCGGCTCTTCCCTTTAACTTCCAGGCTGCC
TATGGCCTGAGTGACCAACTGGCCCAAGCCATCAGTGACCACTATCCAGTGGAGGTGATGCTGA
AGTGAGGCGCGCCTTGCCTAGAAGGCCGGAAACCAACTTTATTACTATATTCCCCACAACCCCC
CTCTCCCTCCCCCCCTAACGTTACTGGCCGAAGCCGCTTGGAATAAGGCCGGTGTGCGTTTGTC
TATATGTTATTTTCCACCATATTGCCGTCTTTTGGCAATGTGAGGGCCCGGAAACCTGGCCCTG
TCTTCTTGACGAGCATTCCTAGGGGTCTTTCCCCTCTCGCCAAAGGAATGCAAGGTCTGTTGAA
TGTCGTGAAGGAAGCAGTTCCTCTGGAAGCTTCTTGAAGACAAACAACGTCTGTAGCGACCCTT
TGCAGGCAGCGGAACCCCCCACCTGGCGACAGGTGCCTCTGCGGCCAAAAGCCACGTGTATAAG
ATACACCTGCAAAGGCGGCACAACCCCAGTGCCACGTTGTGAGTTGGATAGTTGTGGAAAGAGT
CAAATGGCTCTCCTCAAGCGTATTCAACAAGGGGCTGAAGGATGCCCAGAAGGTACCCCATTGT
ATGGGATCTGATCTGGGGCCTCGGTGCACATGCTTTACATGTGTTTAGTCGAGGTTAAAAAACG
TCTAGGCCCCCCGAACCACGGGGACGTGGTTTTCCTTTGAAAAACACGATGATAATAGACGCTA
CGGACTTAAATAATTGAGCCTTAAAGAAGAAATTCTTTAAGTGGATGCTCTCAAACTCAGGGAA
ACCTAAATCTAGTTATAGACAAGGCAATCCTGAGCCAAGCCGAAGTAGTAATTAGTAATGGCTC
CTCACAGAGGCCACCTTCCAGGACTACTCTCTGA

The nucleotide positions of the components in the construct are shown in Table 5 below.

TABLE 5

| Components | Nucleotides |
|---|---|
| 5′ homology arm | 34-73 |
| 3′ self-splicing intron | 74-255 |
| NLS | 256-279 |
| Payload | 280-1062 |
| Spacer | 1086-1108 |
| IRES | 1109-1689 |
| 5′ self-splicing intron | 1690-1809 |
| 3′ homology arm | 1810-1859 |

### Example 5

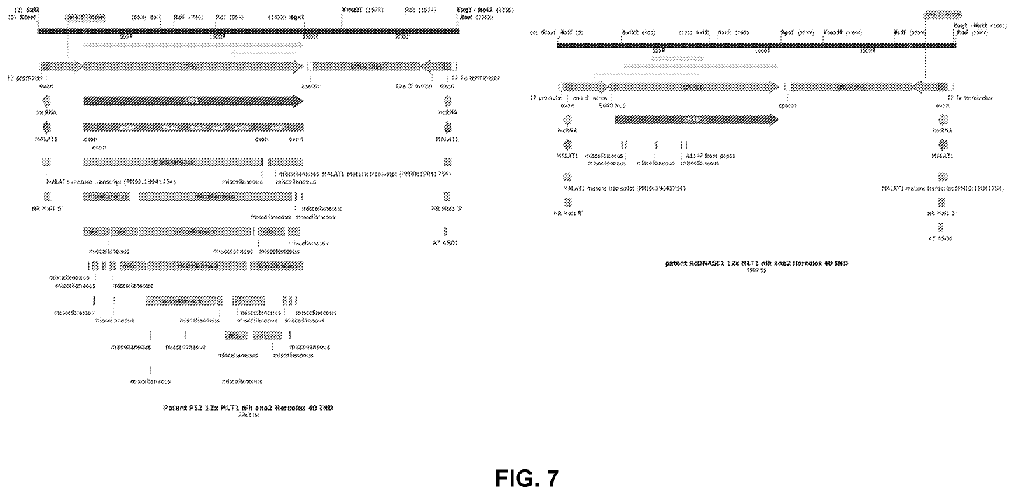

The following Example illustrates the DNA sequence of a DNA construct that produces a linear RNA encoding an rDNAse which is expressed upon circularization of the RNA. The rDNAse represses Metastatic Enriched Apoptosis Induction of Metastatic Cancer cells using Malat1 as target RNA. The DNA constructs, linear RNA, protein products produced on circularization of the linear RNA, and overall experiment is illustrated graphically in FIG. 7.

SEQ ID NO: 5: Malat1 rDNAse
GGGAACACAACTTTTAATCATACTCCAGTCGTTTCACAATAACAATAGATGACTTACAACTAAT
CGGAAGGTGCAGAGACTCGACGGGAGCTACCCTAACGTCAAGACGAGGGTAAAGAGAGAGTCCA
ATTCTCAAAGCCAATAGGCAGTAGCGAAAGCTGCAAGAGAATGAAAATCCGTTGACCTTAAACG
GTCGTGTGGGTTCAAGTCCCTCCACCCCCACCCCCCAAGAAGAAGAGAAAGGTGCTGAAGATCG
CAGCCTTCAACATCCAGACATTTGGGGAGACCAAGATGTCCAATGCCACCCTCGTCAGCTACAT
TGTGCAGATCCTGAGCCGCTATGACATCGCCCTGGTCCAGGAGGTCAGAGACAGCCACCTGACT
GCCGTGGGGAAGCTGCTGGACAACCTCAATCAGGATGCACCAGACACCTATCACTACGTGGTCA
GTGAGCCACTGGGACGGAACAGCTATAAGGAGCGCTACCTGTTCGTGTACAGGCCTGACCAGGT
GTCTGCGGTGGACAGCTACTACTACGATGATGGCTGCGAGCCCTGCGGGAACGACACCTTCAAC
CGAGAGCCATTCATTGTCAGGTTCTTCTCCCGGTTCACAGAGGTCAGGGAGTTTGCCATTGTTC
CCCTGCATGCGGCCCCGGGGGACGCAGTAGCCGAGATCGACGCTCTCTATGACGTCTACCTGGA
TGTCCAAGAGAAATGGGGCTTGGAGGACGTCATGTTGATGGGCGACTTCAATGCGGGCTGCAGC
TATGTGAGACCCTCCCAGTGGTCATCCATCCGCCTGTGGACAAGCCCCACCTTCCAGTGGCTGA
TCCCCGACAGCGCTGACACCACAGCTACACCCACGCACTGTGCCTATGACAGGATCGTGGTTGC
AGGGATGCTGCTCCGAGGCGCCGTTGTTCCCGACTCGGCTCTTCCCTTTAACTTCCAGGCTGCC
TATGGCCTGAGTGACCAACTGGCCCAAGCCATCAGTGACCACTATCCAGTGGAGGTGATGCTGA
AGTGAGGCGCGCCTTGCCTAGAAGGCCGGAAACCAACTTTATTACTATATTCCCCACAACCCCC
CTCTCCCTCCCCCCCTAACGTTACTGGCCGAAGCCGCTTGGAATAAGGCCGGTGTGCGTTTGTC
TATATGTTATTTTCCACCATATTGCCGTCTTTTGGCAATGTGAGGGCCCGGAAACCTGGCCCTG
TCTTCTTGACGAGCATTCCTAGGGGTCTTTCCCCTCTCGCCAAAGGAATGCAAGGTCTGTTGAA
TGTCGTGAAGGAAGCAGTTCCTCTGGAAGCTTCTTGAAGACAAACAACGTCTGTAGCGACCCTT
TGCAGGCAGCGGAACCCCCCACCTGGCGACAGGTGCCTCTGCGGCCAAAAGCCACGTGTATAAG
ATACACCTGCAAAGGCGGCACAACCCCAGTGCCACGTTGTGAGTTGGATAGTTGTGGAAAGAGT
CAAATGGCTCTCCTCAAGCGTATTCAACAAGGGGCTGAAGGATGCCCAGAAGGTACCCCATTGT
ATGGGATCTGATCTGGGGCCTCGGTGCACATGCTTTACATGTGTTTAGTCGAGGTTAAAAAACG
TCTAGGCCCCCCGAACCACGGGGACGTGGTTTTCCTTTGAAAAACACGATGATAATAGACGCTA
CGGACTTAAATAATTGAGCCTTAAAGAAGAAATTCTTTAAGTGGATGCTCTCAAACTCAGGGAA
ACCTAAATCTAGTTATAGACAAGGCAATCCTGAGCCAAGCCGAAGTAGTAATTAGTAAGCATTC
TAATAGCAGCGGGATCAGAACAGTACTGCATTTA

The nucleotide positions of the components in the construct are shown in Table 6 below.

TABLE 6

| Components | Nucleotides |
|---|---|
| 5′ homology arm | 27-66 |
| 3′ self-splicing intron | 67-248 |
| Spacer | 249-272 |
| Payload | 273-1055 |
| 3′ spacer | 1079-1111 |
| IRES | 1112-1682 |
| 5′ self-splicing intron | 1683-1812 |
| 3′ homology arm | 1813-1852 |

### Example 6

In this example, a linear RNA polynucleotide is designed to replace the mutated Huntingtin (HTT) gene in neurons with a wild type version of HTT gene, for the treatment of Huntington's Disease (HD). HD is an inherited disorder caused by a mutation in the HTT gene. This mutation is a CAG repeat expansion in the gene that leads to the production of a mutant Huntingtin protein with an extended polyglutamine stretch, which forms toxic aggregates in neurons leading to progressive neurological symptoms including uncontrolled movements, cognitive problems, and psychiatric disorders.

The linear RNA polynucleotide comprises 5′ and 3′ homology arms for targeting the mutated HTT mRNA that is produced in the neurons of patients with Huntington's Disease. The linear RNA is designed to specifically target the 5′ end of the mutated HTT mRNA, effectively inhibiting the translation of the toxic mutant Huntingtin protein.

The linear RNA polynucleotide comprises a payload RNA, which comprises the coding sequence for the wild type HTT protein, free of the CAG repeat expansion. Once the linear RNA polynucleotide has hybridized to the mutated HTT mRNA and circularized, the payload RNA is translated into the wild type, non-toxic Huntingtin protein.

This linear RNA polynucleotide-based therapy effectively reduces the amount of toxic Huntingtin protein in the neurons, while increasing the levels of non-toxic, functional Huntingtin protein. By replacing the mutant protein with its functional counterpart, the linear RNA polynucleotide alleviates the symptoms of Huntington's Disease and halts or slows the progression of this currently incurable disease.

This example highlights the versatility of linear RNA polynucleotides described herein in addressing genetic diseases, even those caused by complex repeat expansion mutations and manifesting in highly specialized cell types such as neurons.

### Example 7

This example demonstrates a linear RNA polynucleotide for the treatment of Sickle Cell Anemia (SCA). SCA is an autosomal recessive genetic disorder caused by a single base mutation in the beta-globin gene (HBB). This mutation results in the production of an abnormal version of beta-globin called hemoglobin S (HbS). Under low-oxygen conditions, HbS can polymerize, causing the red blood cells to adopt a sickle shape, leading to painful crises, organ damage, and increased risk of infections.

The linear RNA polynucleotide comprises 5′ and 3′ homology arms for targeting the mutated HBB mRNA produced in the red blood cell precursors of patients with SCA. The linear RNA polynucleotide targets the 5′ end of the mutated HBB mRNA, thereby inhibiting the translation of the abnormal HbS protein.

The linear RNA polynucleotide comprises a payload RNA with the coding sequence for the wild type HBB protein. Once the linear RNA polynucleotide hybridizes to the mutated HBB mRNA and circularizes, the payload RNA is translated into the wild-type, functional HBB protein, which forms part of the normal adult hemoglobin (HbA).

With this approach, the linear RNA polynucleotide decreases the production of HbS while augmenting the levels of normal HbA, thereby reducing the sickling of red blood cells. This alleviates the painful symptoms and decrease the organ damage associated with SCA.

This example demonstrates the application of the linear RNA polynucleotides described herein in targeting monogenic diseases caused by single base mutations, with the capability to replace a dysfunctional protein with its functional counterpart, leading to potential symptomatic relief and disease modification.

### Example 8

This example demonstrates using linear RNA polynucleotides for delivering a therapeutic protein selectively in malignant cancer cells, minimizing any potential off-target effects in normal prostate cells or in other tissues.

Two linear RNA polynucleotides are used, each with 5′ and 3′ homology arms for targeting Prostate Specific Antigen (PSA) mRNA and Prostate Specific Membrane Antigen (PSMA) mRNA, respectively. These two target mRNAs are often overexpressed in prostate cancer cells but may also be present at lower levels in normal prostate cells and other tissues.

Both the two linear RNA polynucleotides comprise the same payload RNA with the coding sequence for the pro-apoptotic protein, HSV-TK (herpes simplex virus-thymidine kinase). HSV-TK converts the prodrug ganciclovir into a toxic metabolite, leading to the selective destruction of cells expressing HSV-TK.

Simultaneous delivery of the two linear RNA polynucleotides to a patient increases the therapeutic selectivity for prostate cancer cells and mitigate potential off-target effects, enhancing the safety and efficacy of the therapy. This approach exemplifies the versatility and precision of linear RNA polynucleotides herein in addressing complex and heterogeneous disease conditions like cancer.

### Example 9

A linear RNA polynucleotide was desired to target the prostate specific antigen (PSA) mRNA. The sequence of the linear RNA polynucleotide is set forth in SEQ ID NO: 3.

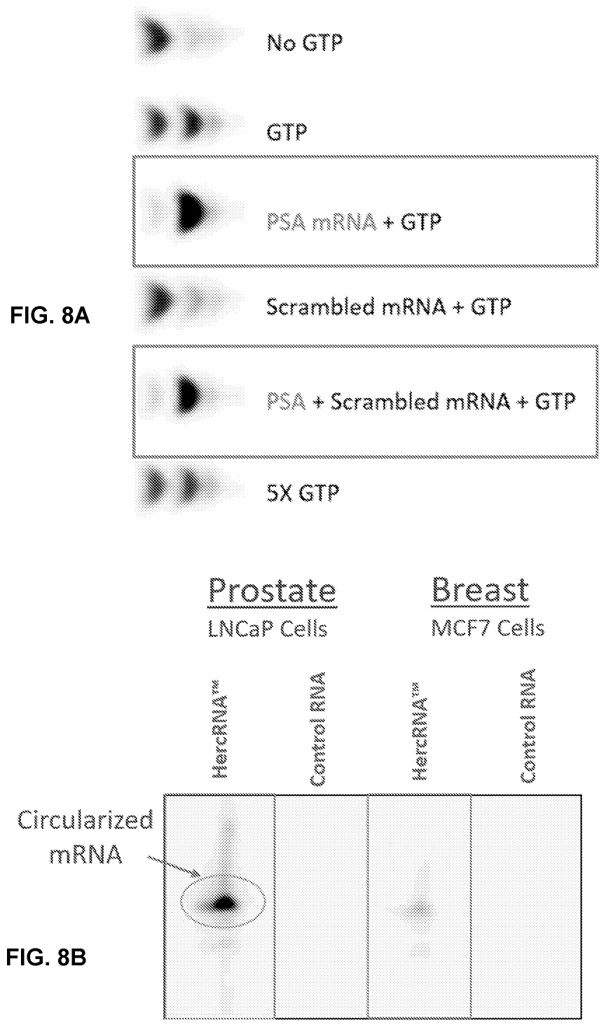

FIG. 8A shows that the linear RNA circularized in the presence of the PSA mRNA. When transfected to prostate cancer cells (LNCaP cells) and breast cancer cells (MCF7 cells), the linear RNA selectively circularized in the prostate cancer cells (cells with high level PSA expression), suggesting PSA-dependent circularization of the linear RNA (FIG. 8B).

While certain embodiments of the present invention have been shown and described herein, it will be obvious to ordinarily skilled artisans that these embodiments are merely exemplary. Numerous variations, changes, and substitutions will occur to ordinarily skilled artisans within the scope and spirit of the invention. Various alternatives to the described embodiments may be employed. Accordingly, the invention should be considered as limited only by the scope of the following claims, and that methods and structures within the scope of these claims and their equivalents are covered.

What is claimed is:

1. A linear RNA polynucleotide for the targeted expression of a payload, the polynucleotide comprising, in the following order:
(i) a 5′ homology arm,
(ii) a 3′ self-splicing intron fragment containing a 3′ splice site dinucleotide,
(iii) optionally, a 5′ spacer sequence,
(iv) a payload sequence,
(v) optionally, a 3′ spacer sequence,
(vi) a 5′ self-splicing intron fragment containing a 5′ splice site dinucleotide, and
(vii) a 3′ homology arm,
wherein the 5′ homology arm and the 3′ homology are complementary to target sequences in a target RNA and are not complementary to each other; and
wherein binding of the homology arms to the target sequences allows splicing/ligation at the 3′ and 5′ splice site dinucleotides and production of a circular RNA in a cell or tissue comprising the target RNA.

2. The linear RNA polynucleotide of claim 1, wherein the self-splicing intron is selected from a group I self-splicing intron, a group II self-splicing intron, and a hammerhead ribozyme.

3. The linear RNA polynucleotide of claim 1 or claim 2, wherein the linear RNA polynucleotide comprises a 5′ spacer sequence, a 3′ spacer sequence, or a 5′ spacer sequence and a 3′ spacer sequence.

4. The linear RNA polynucleotide of any one of the preceding claims, wherein the target RNA is a non-coding RNA.

5. The linear RNA polynucleotide of any one of claims 1-3, wherein the target RNA is a coding RNA, i.e., an mRNA.

6. The linear RNA polynucleotide of claim 4, wherein the target RNA encodes MALAT1.

7. The linear RNA polynucleotide of claim 5, wherein a target sequence is located in the 3′ UTR of a mRNA, or overlaps with the 3′ UTR of the mRNA.

8. The linear RNA polynucleotide of claim 4, wherein a target sequence is located in the 5′ UTR of a mRNA, or overlaps with the 5′UTR of the mRNA.

9. The linear RNA polynucleotide of claim 4, wherein a target sequence comprises, or overlaps with, a start site of the mRNA.

10. The linear RNA polynucleotide of any one of claims 1 to 8, wherein the payload sequence encodes one or more proteins, and the linear polynucleotide further comprises at least one regulatory element downstream of the payload sequence; preferably wherein the at least one regulatory element comprises an IRES.

11. The linear RNA polynucleotide of claim 10, wherein the payload sequence encodes a plurality of therapeutic or diagnostic proteins; optionally wherein the payload sequence further comprises protease cleavage sites between the coding sequences for the plurality of proteins.

12. The linear RNA polynucleotide of claim 11, wherein the therapeutic protein is useful in the treatment of a disease and/or infection, related to a protein deficiency, or able to elicit an immune response for prevention or treatment of disease and/or infection.

13. The linear RNA polynucleotide of claim 12, wherein the therapeutic protein is selected from the group consisting of insulin, GLP-1, adalimumab (HUMIRA®), ghrelin, leptin, an alcohol dehydrogenase or other detoxifying enzyme, a cytokine, an anti-microbial protein, a chemokine, a mitogen, an immunogen, a growth factor, and a differentiation factor.

14. The linear RNA polynucleotide of any one of claims 10 to 13, further comprising a muting sequence that inhibits circularization of the linear RNA polynucleotide in absence of the target RNA; and wherein said inhibition of circularization is relieved in the presence of the target RNA.

15. The linear RNA polynucleotide of any one of claims 10-14, wherein function of the IRES is under control of a riboregulator or riboswitch, preferably wherein said IRES is under control of a riboregulator, more preferably wherein said riboregulator is a toehold riboregulator.

16. The linear RNA polynucleotide of any one of claims 10 to 15, wherein the target sequences are located in the 3′ UTR of an actin RNA and the payload sequence encodes Oct4, Sox2, or Klf4.

17. The linear RNA polynucleotide of any one of claims 10 to 15, wherein the target sequences are located on a Malat1 RNA and the payload sequence encodes a p53 protein.

18. The linear RNA polynucleotide of any one of claims 10 to 15, wherein the target sequences are located in the 5′ UTR, translation start site, or coding region of a defective or mutated RNA, and the payload sequence encodes a functional variant of the defective or mutated RNA.

19. The linear RNA polynucleotide of claim 18, wherein the defective or mutated RNA is a deficient Factor VIII mRNA, a deficient Factor IX mRNA, a deficient CFTR mRNA, a deficient dystrophin mRNA, a deficient insulin mRNA, a deficient Alpha-1 antitrypsin mRNA, a mutated HTT mRNA, or a mutated HBB mRNA.

20. The linear RNA polynucleotide of any of the preceding claims, further comprising a nucleic acid binding peptide ligand sequence; optionally wherein the nucleic acid binding peptide ligand sequence is an MS2 ligand sequence.

21. A pharmaceutical composition comprising one or a plurality of the linear RNA polynucleotide of any one of claims 1 to 20 in a pharmaceutically acceptable vehicle.

22. A DNA construct for production of a linear RNA polynucleotide of any one of claims 1 to 20 comprising, operably-linked in the following order:
(i) a transcriptional promoter, optionally wherein the transcriptional promoter is a bacteriophage T7 promoter;
(ii) a 5′ homology arm,
(iii) a 3′ self-splicing intron fragment containing a 3′ splice site dinucleotide,
(iv) optionally, a 5′ spacer sequence,
(v) a payload sequence,
(vi) optionally, a 3′ spacer sequence,
(vii) a 5′ self-splicing intron fragment containing a 5′ splice site dinucleotide, and
(viii) a 3′ homology arm,
wherein the 5′ homology arm and the 3′ homology are complementary to target sequences in a target RNA and are not complementary to each other; and
(ix) a transcriptional terminator; optionally wherein the transcriptional terminator is a bacteriophage T7 terminator.

23. The DNA construct of claim 22, wherein the self-splicing intron is selected from a group I self-splicing intron, a group II self-splicing intron, and a hammerhead ribozyme.

24. The DNA construct of claim 22, wherein the payload sequence encodes one or more proteins; and the linear polynucleotide further comprises at least one regulatory element downstream of the payload sequence; preferably wherein the at least one regulatory element comprises an IRES.

25. The DNA construct of claim 24, wherein the payload sequence encodes a plurality of proteins; optionally wherein the payload sequence further comprises protease cleavage sites between the coding sequences for the plurality of proteins.

26. A method of cell specific expression of a payload sequence in a target cell or tissue, the method comprising: delivering to the target cell or target tissue the linear RNA polynucleotide of any one of claims 1-20, or the pharmaceutical composition of claim 21, wherein the target cell or target tissue expresses a cell specific target RNA that comprises a target sequence.

27. The method of claim 26, wherein the payload sequence encodes one or more proteins, and the linear polynucleotide further comprises an IRES downstream of the payload sequence, and wherein circularization of the linear RNA polynucleotide places/relocates the IRES to a position upstream (5′) of the payload sequence and permits translation of the protein.

28. The method of claim 26, wherein the linear RNA polynucleotide is delivered to the cell in vivo.

29. The method of claim 26, wherein the linear RNA polynucleotide is delivered to the cell in vitro.

30. The method of claim 26, wherein the linear RNA polynucleotide is transfected into the cell by electroporation or lipofection.

31. The method of claim 26, wherein the linear RNA polynucleotide is transfected into the cell using a delivery vehicle.

32. The method of claim 26, wherein the delivery vehicle is a polymeric carrier; an exosome; a lipid carrier; and/or a lipid nanoparticle.

33. The method of any one of claims 26-32, wherein the linear RNA polynucleotide comprises a nucleic acid binding peptide ligand sequence; optionally wherein the nucleic acid binding peptide ligand sequence is an MS2 ligand sequence, and the delivery vehicle is a yeast cell.

34. A method for treating a disease or disorder, the method comprising delivering a payload sequence encoding one or more therapeutic proteins to a target cell or tissue, wherein the payload sequence is present on a linear RNA polynucleotide comprising, in the following order:
(i) a 5′ homology arm,
(ii) a 3′ self-splicing intron fragment containing a 3′ splice site dinucleotide,
(iii) optionally, a 5′ spacer sequence,
(iv) the payload sequence,
(v) optionally, a 3′ spacer sequence,
(vi) a 5′ self-splicing intron fragment containing a 5′ splice site dinucleotide, and
(vii) a 3′ homology arm,
wherein the 5′ homology arm and the 3′ homology arm are complementary to target sequences in a target RNA and are not complementary to each other; and
wherein binding of the homology arms to the target sequence allows splicing/ligation at the 3′ and 5′ splice site dinucleotides and production of a circular RNA in a cell or tissue comprising the target RNA.

35. The method of claim 34, wherein the linear RNA polynucleotide further comprises an IRES downstream of the payload sequence, and wherein circularization of the linear RNA polynucleotide places/relocates the IRES to a position upstream (5′) of the payload sequence and permits translation of the protein.

36. The method of claim 32 or claim 33, wherein the linear RNA polynucleotide further comprises a muting sequence that inhibits the production of the circular RNA in a cell or tissue lacking the target RNA.

37. The method of any one of claims 33-34, wherein the linear RNA polynucleotide further comprises a riboregulator or riboswitch that functions to selectively inhibit or allow translation of the payload sequence.

38. The method of any one of claims 26-37, further comprising delivering to the target cell or tissue a second linear RNA polynucleotide comprising the same payload sequence but different 5′ and 3′ homology arms complementary to a different target sequence.

39. The method according to any one of claims 26-37, wherein the payload sequence encodes a plurality of therapeutic proteins; optionally wherein the payload sequence further comprises protease cleavage sites between coding sequences for each protein.
